(19) United States
(12) Patent Application Publication
(10) Pub. No.: US 2003/0143558 A1
(43) Pub. Date: Jul. 31, 2003

(54) Methods for attenuation of virulence in bacteria

(75) Inventors: Wayne Mitchell, San Francisco, CA (US); Adam Cota, Berkeley, CA (US); T. Guy Robert, Oakland, CA (US)

Correspondence Address: QUINE INTELLECTUAL PROPERTY LAW GROUP, P.C., P O BOX 458, ALAMEDA, CA, 94501, US

(73) Assignee: Tao Biosciences, LLC

(21) Appl. No.: 10/157,736

(22) Filed: May 28, 2002

Related U.S. Application Data
(60) Provisional application No. 60/293,770, filed on May 25, 2001.

(51) Int. Cl. C12Q 1/68; G06F 19/00; G01N 33/48; G01N 33/50

(52) U.S. Cl. 435/6; 702/20

(57) ABSTRACT

The present invention provides methods of putatively identifying, based on presence of rare codon usage, cellular components involved in virulence. Also included are methods of verifying putative virulence genes and methods of attenuating such virulence, e.g., through identification and modification of genes/gene products that modulate translation of gene subsets involved in pathogen virulence. The methods include examining the codon usage and frequency employed in the organism, and identifying and structurally characterizing, e.g., tRNA molecules associated with over-represented or under-represented codons. By targeting the cell's ability to decode specific sets of genes, the virulence of a pathogen can be modulated.

### CROSS REFERENCE TO RELATED APPLICATIONS

Pursuant to 35 USC §19(e), this application claims priority to, and benefit of, U.S. Provisional Patent Application Serial No. 60/293,770, filed on May 25, 2001, the disclosure of which is incorporated herein in its entirety for all purposes.

### FIELD OF THE INVENTION

The invention relates to the field of detection and attenuation of virulence of pathogenic organisms (typically bacteria). In particular, the invention relates to determination of the occurrence of rare codon usage in genes associated with an organism's pathogenicity, e.g., virulence genes located in “pathogenicity islands.” The invention also provides methods for using such determination of rare codon usage in methods to identify genes involved in a pathogen's virulence and to attenuate the virulence of the pathogenic organism through identification of, and use of, virulence modulating compounds. Also included are computer systems, compositions, kits and screening systems incorporating aspects of the invention.

### BACKGROUND OF THE INVENTION

Bacterial pathogens are a varied set of bacteria that cause a wide variety of diseases in humans, plants, and animals. The genetic elements which give rise to pathogenicity are similarly varied and are often mobile. See, e.g., Hacker, J., et al. (1999) “Pathogenicity Islands and Other Mobile Virulence Elements” American Society for Microbiology, Washington D.C., pp. 1-11. For example, pathogenicity plasmids are capable of being transferred from one bacteria to another. See, e.g., Sansonetti, P. et al. (1983) Infect Immun 39(3):1392-1402. Additionally, chromosomally encoded loci giving rise to pathogenicity are often encoded within mobile regions flanked by IS elements, tRNAs or transposons. See, e.g., Bach, S. et al. (2000) FEMS Microbiology Letters, 183:289-294; Censini, S. et al. (1996) Proc Natl Acad Sci USA 93:14648-14653; and Blum, G. et al., (1994) Infection and Immunity 62:606-614. Other genes which confer pathogenicity may be contained with the DNA of a bacteriophage. See, e.g., Nakayama, K. et al., (1999) Molecular Microbiology 31:399-419 and Plunkett, G., et al., (1999) J Bacteriol 181(6):1767-78.

Pathogenicity islands (PAIs) are regions present in some bacterial strains, which contain several genes involved in virulence and which are absent from nonpathogenic strains. These regions can vary in size from about 1.5 kb to over 200 kb in size, and may be mobile. PAls are often inserted into the 3′ end of tRNA genes within the bacterial genome, and like many plasmids, may exhibit codon usage patterns and G+C contents which differ from those of the host bacteria. See, e.g., Hacker, supra. Such islands are part of a broader group of genetic elements known as genomic islands which encode sequences relating to, e.g., pathogenicity, fitness, symbiosis, and resistance, etc.

A welcome addition to the art would be the ability to identify specific genes or sequences involved in pathogenicity (such as virulence), as well as methods using such identification to attenuate the virulence/pathogenicity of bacterial strains carrying the specific genes. The present invention provides these and other benefits which will be apparent upon examination of the following specification and figures.

### SUMMARY OF THE INVENTION

The invention provides methods and compositions for detection and attenuation of virulence of pathogenic organisms (typically bacteria). More specifically, the invention provides methods to determine rare codon usage in genes involved with an organism's pathogenicity (e.g., virulence genes located in pathogenicity islands). The invention also provides methods for using the determination of virulence of identified genes comprising rare codon usage and methods for attenuation of virulence of the organism through modification of one or more identified gene (and/or gene product) comprising the rare codon usage (and/or modification of one or more gene or gene product which interacts with or modifies the identified gene comprising the rare codon usage). The invention also provides methods of screening for identification of areas of rare codon usage in genes involved in pathogenesis/virulence and methods of identification of compounds (e.g., enzymes, proteins, chemical compounds, ribozymes, etc.) that effect virulence of genes and/or gene products identified through analysis of rare codon usage, etc. Also included are computer systems, compositions, kits and screening systems incorporating aspects of the invention, etc.

In some aspects the present invention comprises a method of determining a difference in codon usage between a selected nucleic acid sequence and a reference genome, comprising: (a) selecting a codon ‘i’ from a set of ‘n’ codons; (b) determining the number of occurrences of the codon i in the selected nucleic acid sequence and also in the reference genome; (c) calculating a first occurrence frequency ‘fᵢ’ by determining fᵢ=(#codonᵢ)(1000 codons)/(#codons in all reference genome open reading frames, ORFs); (d) calculating a second occurrence frequency ‘cᵢ’ wherein cᵢ=(#codonᵢ) (1000codons)/(#codons in the selected sequence or in the selected open reading frame, ORF); and (e) calculating an average difference CDI between the first occurrence frequency (or fᵢ) and the second occurrence frequency (or cᵢ), wherein

$$CDI = \frac{{\sum }_{i - 1}^{n} \vert {c}_{i} - {f}_{i} \vert}{n}$$

and wherein the value of CDI indicates the difference in usage of the particular codon in a selected sequence and the usage of the particular codon in the reference genome. In some embodiments, the set ‘n’ of codons comprises the common 61 non-stop codons. In other embodiments, the set ‘n’ comprises a subset of the 61 non-stop codons (e.g., a set of rare codons, the 10 rarest codons in the reference genome, etc.), the common 64 codons including stop codons, etc. In some embodiments, the first occurrence frequency ‘fᵢ’ and/or the second occurrence frequency, ‘cᵢ’ is calculated only with reference to open reading frames (ORFs) that are greater than about 250 or more amino acids in length.

In other aspects, the current invention comprises a method of identifying a putative target for attenuation of pathogen virulence through (a) determining a codon usage frequency of one or more codon of a pathogen; (b) identifying at least one gene comprising one or more over-represented codon or one or more under-represented codon (e.g., wherein such codons are rare usage codons); (c) identifying a set of tRNA molecules responsible for interacting with the one or more over-represented (optionally rare usage) codon or under-represented (rare usage) codon in the at least one gene during translation; (d) providing a population of nucleic acid sequences encoding a putative target for attenuation of pathogenic virulence and an in vitro or in vivo translation system; (e) altering a translation process involving one or more member of the set of tRNA molecules and the in vitro or in vivo translation system, thereby altering expression of at least one member of the population in (d); and (f) testing for one or more effect of the altering, thereby identifying one or more putative target for attenuation of pathogen virulence. In some embodiments, the altering of the translation process comprises preventing the one or more members of the set of tRNA molecules from interacting with an mRNA encoding a putative target. In other embodiments, the altering of the translation process comprises interfering with a process for synthesizing one or more members of the set of tRNA molecules (optionally wherein such comprises altering a base modification in the tRNA sequence). In other embodiments, altering the translation process comprises altering the translation efficiency or accuracy of one or more member of the set of tRNA molecules. In some embodiments, the method further comprises screening one more compositions (e.g., various libraries, etc.) for one or more virulence modulatory effect on the target. Such compositions optionally comprise, e.g., 100, 250, 500, 750, 1,000, 2,500, 5,000, 7,500, 10,000 or more compositions within them.

In some aspects, the current invention comprises a method for identifying virulence-related nucleic acid sequences in a pathogenic organism by: (a) analyzing a population of nucleic acid sequences derived from the pathogenic organism and identifying one or more over-represented codons or under-represented codons as compared to a nonpathogenic organism; (b) determining a distribution for at least one member of the one or more over-represent codons or under-represented codons (e.g., in some embodiments such distribution is optionally determined by calculating a distribution value ‘D’ for at least one member of the one or more over/under represented codons, wherein D=(A*1000)/n wherein for each gene/ORF D equals the number of codon type ‘A’ divided by ‘n’ total codons (normalized to per 1000 codons); (c) selecting a subset of nucleic acid sequences from the population of nucleic acid sequences based upon the distribution of the over-represented or under-represented codons; and (d) analyzing the subset of nucleic acid sequences for virulence activity, thereby identifying one or more virulence-related nucleic acid sequence in a pathogenic organism. In some embodiments the subset of nucleic acid sequences is selected based upon a number of over-represented codons in the nucleic acid sequence while in other embodiments, the subset of nucleic acid sequences is selected based upon a number of under-represented codons in that nucleic acid sequence. In other embodiments, the nonpathogenic organism and the pathogenic organism are different serovars of a common ancestral organism or are two strains of the same species. In some embodiments the nonpathogenic organism is E. coli K12 and the pathogenic organism is, e.g., one or more of E. coli 0157:H7, E. coli B171, or Shigella flexneri. For example, although E. coli 0157 (a pathogenic organism) and E. coli K12 (a common lab strain and normal communal in the gut) are both “E. coli” they have a number of differences at the genomic level. For example, E. coli 0157 has greater than 1000 genes that are not present in E. coli K12, plus most of the K12 genes. In other words, 0157 and K12 share about 4500 genes, with 0157 having an additional approximate 1000 genes that are not present in K12. Thus the “control” genes are the shared genes that have a codon usage distinct from the 0157 specific “shared” genes. Thus, in optional embodiments in attenuation of virulence herein there must be virulence specific genes that have a distinct codon usage. In other embodiments the method of identifying virulence related nucleic acid sequences in a pathogenic organism comprises wherein the virulence related nucleic acid sequence comprises one or more tRNA molecule responsible for encoding the at least one member of the one or more over-represented or under-represented codon (e.g., the rare usage codon that is over/under represented in that gene as compared to its usage in the rest of the genome). In some embodiments, such method further comprises identifying one or more structural characteristics of the one or more tRNA molecule and modulating the activity of the one or more tRNA molecule. In some embodiments, the virulence-related nucleic acid sequence comprises one or more tRNA synthase molecule and optionally can further comprise: identifying one or more structural characteristic of the one or more tRNA synthase molecule and modulating the activity of such molecule. In some embodiments, the method further comprises screening one more compositions (e.g., various libraries, etc.) for, e.g., one or more virulence-related nucleic acid sequences. Such compositions optionally comprise, e.g., 100, 250, 500, 750, 1,000, 2,500, 5,000, 7,500, 10,000 or more compositions within them.

In yet other aspects, the current invention comprises a method of regulating gene expression in a bacterial organism by: (a) identifying one or more over-represented codon or under-represented codon within a set of nucleic acid sequences from a bacterial organism; (b) identifying at least one tRNA species responsible for encoding at least one of the one or more over-represented or under represented codon; and (c) modulating an expression or activity of the at least one tRNA species in the bacterial organism, thus, altering a translation of a nucleic acid sequence comprising the one or more over represented or under represented codon, thereby regulating the expression of one or more gene in the bacterial organism. In some embodiments, the identifying of the one or more over-represented codon or under represented codon comprises determining a distribution for at least one member of the one or more over-represented codons or under represented codons (e.g., in some embodiments such distribution is optionally determined by calculating a distribution value ‘D’ for at least one member of the one or more over/under represented codons, wherein D=(A*1000)/n wherein for each gene/ORF D equals the number of codon type ‘A’ divided by ‘n’ total codons (normalized to per 1000 codons). In other embodiments, the set of nucleic acid sequences from the bacterial organism comprises a library of mRNA sequences. In yet other embodiments, the set of nucleic acid sequences from the bacterial organism comprises sequences from one or more pathogenicity islands. In other embodiments, the identifying of the at least one tRNA species comprises: (a) measuring the codon usage of each gene in the bacterial organism (optionally wherein the measuring comprises use of a counting algorithm, optionally in PERL language code); (b) cataloging the at least one tRNA genes in the bacterial organism (optionally done with tRNAscan-SE software); and (c) detecting one or more modification in the tRNA which will modulate expression of one or more gene in the bacterial genome wherein the one or more gene is rich in a particular codon (optionally wherein such detecting is based on cognate codon-anticodon interactions and/or codon-anticodon wobble rules). In some embodiments herein, modulating the expression or activity of the at least one tRNA species comprises altering a chemical character or chemical characteristic of the tRNA species. Some embodiments herein also include wherein modulating the expression or activity of the at least one tRNA species comprises reducing an extent of diversity of the tRNA species (e.g., making the unmodified tRNA only and/or not allowing any rare-coding-encoding activity). Still other embodiments include wherein modulating the expression or activity of the at least one tRNA species comprises inhibiting a tRNA modification synthase activity specific for that at least one tRNA species. In other words, not enough (or any) of the functional, modified tRNA species is made/present. Thus growth is inhibited, etc. For example, E. coli 0157 virulence genes are very rich in the rare isoleucine codon AUA which is translated by a modified tRNA (the lysidine modification, see below). Thus, if there is an inhibitor that is only a partial inhibitor of the tRNA lysidine synthase, it is optionally able to reduce lysidine modification (e.g., reduced by one half, etc.). Thus, such optionally stops translation of the genes that are richest in AUA codons (e.g., the virulence genes), but would not stop all translation of all AUA genes. Thus with E. coli 0157, such would result in the suppression of the AUA rich pathogenicity genes, resulting in a bacteria that will live fine, e.g., in a subject's gut, but which would not be able to initiate intracellular invasion, etc. (e.g., the virulence actions which require the pathogenicity genes). Other embodiments herein include wherein modulating the expression or activity of the at least one tRNA species comprises inhibiting an interaction between the tRNA species and an additional RNA molecule (e.g., an mRNA molecule, an rRNA molecule, a tmRNA molecule, an snoRNA molecule, or other RNA or ribonucleic/protein particle, etc., optionally after making an inappropriate modification or no modification to the tRNA). Other embodiments include wherein the activity of the tRNA is altered by modulating the extent of modification of the tRNA (especially because only the properly modified tRNA is functional and/or completely or correctly functional). Other embodiments include wherein altering the translation of the nucleic acid sequence comprises inhibiting the translation of an mRNA molecule or enhancing the translation of an mRNA molecule (e.g., optionally thus reducing availability of rare-codon-encoding tRNA). In some embodiments, the method further comprises screening one more compositions (e.g., various libraries, etc.) for, e.g., one or more compound that modulates expression or activity of the at least one tRNA species. Such compositions optionally comprise, e.g., 100, 250, 500, 750, 1,000, 2,500, 5,000, 7,500, 10,000 or more compositions within them.

In yet other aspects, the current invention comprises a method of attenuating the virulence of a pathogenic organism by (a) identifying one or more tRNA species encoding one or more over represented codon within a set of virulence related nucleic acid sequences from a bacterial organism (wherein the over represented, optionally rare, codon is over represented in relation to a usage of the, optionally rare, codon in the rest of the genome) and (b) inhibiting an in vivo expression or activity of the tRNA species within the bacterial organism, thereby decreasing the virulence of the pathogenic organism. In some embodiments the inhibiting of the in vivo expression or activity of the tRNA species comprises reducing an extent of diversity of the tRNA species. In other embodiments, inhibiting the in vivo expression or activity of the tRNA species comprises inhibiting a tRNA synthase activity specific for the one or more tRNA species. Other embodiments include wherein inhibiting the in vivo expression or activity of the tRNA species comprises inhibiting an interaction between the tRNA species and an additional RNA molecule.

In other aspects, the current invention comprises a method for selectively affecting one or more pathogenic organism in a population, the method comprising (a) providing a first population comprising nucleic acid sequences from a pathogenic organism; (b) providing a second population comprising nucleic acid sequences from a nonpathogenic organism (which optionally is of the same species as the pathogenic organism), (c) determining a distribution of codon usage in the pathogenic organism as compared to a distribution of a codon usage in the nonpathogenic organism; (d) selecting one or more, optionally rare, codon that are over represented or under represented in the nucleic acid sequences of the pathogenic organism based upon the distribution of codon usage in the pathogenic organism and the nonpathogenic organism, (e) identifying at least one tRNA species responsible for encoding at least one selected codon (which selected codon comprises one that is over represented or under represented in the pathogenic organism relative to the nonpathogenic organism, and (f) altering the expression or activity of the identified tRNA species, thereby selectively affecting the pathogenic organisms in the population. In some embodiments, the altering comprises identifying one or more structural characteristics of the at least one tRNA species and providing an antibody specific to the at least one tRNA which binds to the tRNA (thus preventing an action such as involved in translation, etc., by the tRNA). In other embodiments, the altering comprises identifying one or more enzymes for synthesizing the one or more tRNA species and inhibiting such identified synthesizing enzymes.

In yet other aspects, the current invention comprises a method for altering the susceptibility of a mRNA sequence to translation errors. For example, one effect of loss of tRNA modification is translational errors such as, e.g., frame shifting, etc.

In yet other aspects, the current invention comprises a method for selectively expressing proteins. Thus any phenotype associated with genes having a unique codon usage are optionally modulated by this method. For example, an engineered metabolic pathway in a bacterium makes some desirable product. The genes coding for such desirable product are optionally enriched with rare usage codons and the appropriate tRNA modification is used to modulate the expression of such genes. Thus, modulation of the phenotype is optionally as simple as expressing a single protein of interest, in which situation, the method optionally distills to the overexpression of the protein. The invention also includes a method of regulating gene expression in a bacterial organism, the method comprising: a) identifying one or more over or under represented codons within a set of nucleic acid sequences from an organism; b) identifying at least one tRNA species responsible for encoding at least one member of the one or more over/under represented codons; c) modulating an expression or activity of the at least one tRNA species in the organism; and, d) altering a translation of a nucleic acid sequence comprising the one or more over/under represented codons, thereby regulating the expression of one or more genes in the organism wherein in such method, the altering the translation of the nucleic acid sequence comprises enhancing the translation of an mRNA molecule. Thus the goal is this embodiment is to upregulate and enhance desirable molecules (e.g., not only anti-virulence per se, but also actually enhancing desirable products in a natural or engineered stain).

These and other objects and features of the invention will become more fully apparent when the following detailed description is read in conjunction with the accompanying figures.

### BRIEF DESCRIPTION OF THE DRAWINGS

FIG. 1: depicts the G+C content in various virulence elements as compared to the G+C content of the corresponding host organism.

FIG. 2, PANELS A and B: depict CDI (panel A) of E. coli genes larger than 250 amino acids in length and RCDI (panel B) of E. coli genes larger than 250 amino acids in length.

FIG. 3, PANELS A and B: depict CDI (panel A) of P. aeruginosa genes larger than 250 amino acids in length and RCDI (panel B) of P. aeruginosa genes larger than 250 amino acids in length.

FIG. 4: depicts percentage of genes in pathogenicity elements which exceed the 95% CDI/RCDI value for host genome of various species.

FIGS. 5, PANELS A and B: depict ATA (panel A) codon frequency in pO157 Virulence Associated Plasmid Genes and AGG (panel B) codon frequency in genes of E. coli 0157-H7 pO157 pathogenicity plasmid.

FIG. 6: depicts codon frequencies in virF of codons recognized by miaA substrates.

FIG. 7: depicts rare codon usage in virulence genes of pO157 compared to genes of E. coli.

### DETAILED DESCRIPTION

Definitions

Before describing the present invention in detail, it is to be understood that this invention is not limited to particular compositions or biological systems, which can, of course, vary. It is also to be understood that the terminology used herein is for the, purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

Unless defined otherwise, all scientific and technical terms are understood to have the same meaning as commonly used in the art to which they pertain. For the purpose of the present invention, the following terms are defined below.

The term “nucleic acid” as used herein is generally used in its typical art-recognized meaning to refer to a ribose nucleic acid (RNA) or a deoxyribose nucleic acid (DNA) polymer or analog thereof, e.g., a nucleotide polymer comprising modifications of the nucleotides, a peptide nucleic acid (PNA), or the like. In certain applications, the nucleic acid can be a polymer including both RNA and DNA subunits. A nucleic acid can be, e.g., a chromosome or chromosomal segment, a vector (e.g., an expression vector), a naked DNA or RNA polymer, the product of a polymerase chain reaction (PCR), an oligonucleotide, a probe, etc.

The term “serovar,” as used herein, refers to a serological variety of a species (usually a prokaryote) that is characterized by its antigenic properties.

The term “polynucleotide sequence” refers to a contiguous sequence of nucleotides in a single nucleic acid or to a representation, e.g., a character string, thereof, depending on context.

The term “amino acid sequence” refers to a polymer of amino acids (e.g., a protein, polypeptide, etc.) or to a character string representing an amino acid polymer, depending on context.

The term “tRNA” has its common art-related use herein. Thus, tRNA refers to the small RNA molecule (e.g., between about 70 and 90 nucleotides long) which by binding at one position to a specific codon on an mRNA (via interaction between the codon and the corresponding anti-codon on the tRNA) and at another position to an amino acid specified by that specific codon, allows an amino acid to line up according to the sequence of the nucleotides on the mRNA.

As used herein the term “pathogenicity” (and also pathogenic, pathogen, etc. depending upon context) refers to the capacity of an organism (e.g., a bacterium) to cause disease (and/or disease related states or conditions). The term “virulence” is to be taken to be a measure of an organism's pathogenic potential or its pathogenicity. In typical usages herein, such involves, e.g., the presence of specific genes and/or gene products in an organism, e.g., such as those related to gut wall adherence, hemolysis, etc.

A rare codon herein is one that is used infrequently by an organism (e.g., a codon that is not the frequently used codon to correspond to a particular amino acid in that organism and or gene). Thus what constitutes a rare codon varies from organism to organism, (or from one group of genes to another group of genes), etc. Further specific examples are given below. An under-represented codon and an over-represented codon are to be taken to typically mean an under or over represented rare usage codon (e.g., that is under or over represented in one gene/ORF/sequence as compared to its representation in, e.g., the rest of the genome or other comparison sequences). Again, such over or under representation is variable depending upon, e.g., the specific codon usages in the genes and genomes under consideration. As a hypothetical example in a hypothetical genome, where genes range from a codon usage of 1 AUA codon per 1000 codons up to 6 AUA codons per 1000 codons, then the 6 AUA usage is to considered over-represented in such case. As also used herein “rich,” and “enriched” areas (e.g., in terms of codon usage or rare codon usage) are to be taken to be equivalent with over-represented areas.

Bacteriocidal Treatment versus Attenuation of Pathogenic Virulence

The present invention includes identification of specific nucleic acid sequences in pathogenic organisms that can optionally serve as drug targets or which encode products which can optionally be sensitive to drug targets, thus leading to attenuation of virulence of the pathogenic organism. In typical embodiments, the identification includes, e.g., conducting surveys of codon usage in a pathogenic organism of interest, identifying genes that have over-represented or under-represented codons (e.g., genes involved in virulence/pathogenicity); identification of tRNAs responsible for decoding such codons (e.g., those codons with unusual frequencies); and/or identification of characteristics of such tRNAs that can provide targets for inhibitors of the function of those specific tRNAs.

The present invention provides methods of identifying gene sequences (e.g., those involved in virulence and/or pathogenicity) comprising high usage of rare or unusual codons. Control of cellular components that modulate translation of gene subsets involved in such pathogen virulence sequences can allow control over the pathogenic organism, or, more precisely, over the pathogenicity of the organism. The methods, etc., detailed herein, include examining the codon usage and frequency employed in the organism (e.g., identifying rare codon usage and location of such), and then identifying and structurally characterizing the tRNA molecules associated with such rare, or over-represented or under-represented codons. By targeting the cell's ability to decode specific sets of genes (e.g., virulence genes), the virulence of a pathogen can be modulated. Thus, as described herein, the invention comprises novel computational methods for identifying one or more set of proteins that can be co-regulated by targeting the cell's ability to decode these sets of genes, for example, by targeting specific tRNA molecules. The result is that certain phenotypes, including, but not limited to, nutrient dependence, spore formation, secretion, and production of sets of gene products, whether natural or engineered into the bacteria, can be targeted for modulation. In typical embodiments, the gene products are involved in bacterial pathogenicity.

Bacterial mechanisms of pathogenicity are often somewhat distinct from the genetic pathways that support an organism's survival under specific physical conditions. Historically, medical treatments for infection have sought to destroy the invading organisms. However, it is optionally possible to effect a successful treatment without killing the pathogen. In many cases this may be preferable. For example, antibiotic use is often accompanied by disruption of the normal bacterial flora that live as commensal organisms on the surface tissues of human subjects. More than 200 species of bacteria are included in the normal flora, the vast majority of which are found in the gastrointestinal tract. These bacteria bring many benefits to the human host, for example, the synthesis of vitamins K and B12, the formation of biofilms that exclude pathogens, the stimulation of development of immune tissues in the GI tract and the generation of the immune response to invading bacteria.

Thus, the indiscriminate destruction of a bacterial pathogen through use of antibiotic treatment may have adverse effects to the host. Additionally, use of antibiotics may lead to bacterial resistance to the antibiotic. Anti-microbials (e.g., antibiotics) are often associated with patient morbidity, typically in the form of post-treatment diarrhea due to the process of recolonization. One of the best characterized reactions to anti-microbial therapy is due to infection by the adventitious organism, Clostridium difficile. C. difficile is carried among the normal flora in 5-46% of adults and up to 70% of children under 1 year of age. However, the bacterium spreads in the GI tract during therapy with any of several classes of antibiotics (e.g., antibiotics which kill off competing microorganisms in the gut) and produces toxins that cause pathology ranging from mild diarrhea to ulcerative colitis. Such reactions often necessitate withdrawal of antibiotics to allow normal flora to become re-established.

Additionally, adverse reactions are frequently associated with use of antibiotics in high doses. The currently high prevalence of Pseudomonas aeruginosa among patients with cystic fibrosis is thought to have resulted from the use of cephalizin for treatment of pulmonary S. aureus. The high levels of antibiotics used allow P. aeruginosa to reach high levels in the lung sputum and increase the risk of systemic toxicity. Furthermore, antibiotic treatment rarely achieves a 100% kill rate of P. aeruginosa. Such partial killing leads to the development of resistant strains in up to 15% to 100% of patients. Thus, the use of antibiotics presents a double-edged sword.

As can be appreciated, in many cases there are clear advantages to therapies that inhibit pathogenic mechanisms without killing the targeted bacteria. Such ‘anti-virulence’ therapies are optionally designed to limit the pathogenic organism's ability to, e.g., secrete toxins, specifically adhere to host tissues, or perform other functions that lead directly to pathogenesis. The normal growth of the targeted bacteria is optionally not severely impacted. Therefore, the selective pressure produced by antibiotics designed to target essential functions of such bacteria is reduced. For example, for pathogenic strains of E. coli, if modulation of the Type III secretion system is targeted, the organism will likely be less adapted to survival in the gut than the resident commensal bacteria and, thus, will optionally be unable to persist or live in the gut. The current invention allows specific control of virulence/pathogenicity of organisms through attenuation of virulence rather than anti-microbial action. Thus, the invention is especially useful when other methods of controlling such organisms or their pathogenicity (e.g., antibiotics) are not preferred, or are ineffective.

Methods of Attenuation of Bacterial Virulence of the Invention

Identification of the distribution of codons in genes of interest (GOI) and/or identification of genes that contain a particularly high or low frequency of a particular codon (e.g., a rare or rare usage codon) are employed in the methods to effect or modulate bacterial phenotypes (e.g., virulence, etc.).

The present invention provides methods for identifying cellular components that can be manipulated to modulate the expression of specified gene subsets (e.g., those involved in pathogenicity, etc.) at the level of protein translation. For example, components such as tRNAs (and modifications thereof) are optionally manipulated. Various combinations of tRNA and biochemical modifications to the tRNA potentiate the translation of different triplet codons. Each gene that is translated has a definite codon profile. This allows genes to be categorized according to the occurrence of unusual frequencies of one or more codons within the gene. Analysis of these codon profiles and categorized genes leads to the identification of cognate tRNA (and modifications) that exert disproportional influence on the translational expression of such genes. Disruption or enhancement of the activity of such disproportional influencing tRNAs (and/or the modifications therein) will impact translation of the cognate gene subsets.

When genes of interest have an extreme codon frequency (e.g., have a high number of rare codons, etc.) for one or more codons, the tRNA and tRNA modifications responsible for translation of the codons for that profile provide points of intervention whereby expression of those genes can be modulated. Furthermore, since particular genes sometimes themselves modulate cascades of secondary and tertiary gene expression, control of translation of these key genes will also affect the expression of genes not necessarily containing the under- or over-represented codons identified through the methods of the present invention.

Gene subsets of interest typically include those involved in pathogenicity, but may also include, e.g., genes responsible for developmental processes, environmental response, virulence, and the like. Modulation of these genes or gene subsets provides basis for modulation of the corresponding phenotype. Furthermore, the genes or gene subsets are not restricted to naturally occurring genes. One or many genes might be altered prior to introduction into an organism to give the introduced genes a codon frequency profile that places them in (or removes them from) a regulated gene subset. Thus, translation of a new or synthetic gene maybe modulated in a predictable way.

The methods of the present invention optionally include, e.g., the steps of determining the codon usage in a pathogen, and identifying one or more genes having one or more over-represented codons, or one or more under represented codons, e.g., rare usage codons. For example, in one embodiment of the steps involved in the determination of codon usage in a pathogen, a list of gene sequences from a pathogen of interest (or an organism of interest, OII) is input into, for example, a computer, a database, or a spreadsheet. The codons are then tabulated, and their frequency of usage is calculated. The number of each of the 64 naturally occurring codons is counted in each gene. Normally there are 61 codons with UAA, UAG, and UGA being read as stops, e.g., stop codons. However, exceptions to this rule include UGA, which is read as selenocysteine in some genes of some organisms and as trp in mycoplasms. As such, any or all 64 codons are optionally analyzed in the current invention. For each gene, the frequency of each codon (defined as codon number normalized according to gene length) is determined. The data is optionally presented in the form of a table, or matrix, containing the possible codons and their frequency of occurrence per gene or per open reading frame (ORF). For example, the output optionally comprises two 64 by ‘n’ matrices, where n is the number of ORFs; the columns represent the 64 codon frequencies; and there are 64 codon counts for each ORF.

Any of a variety of statistical analysis methods can be used to assess codon frequency. For example, a variety of statistical and other bioinformatics methods that can optionally be applied to the present invention are found in, e.g., Hinchliffe (1996) Modeling Molecular Structures John Wiley and Sons, New York, N.Y.; Gibas and Jambeck (2001) Bioinformatics Computer Skills O'Reilly, Sebastopol, Calif.; Pevzner (2000) Computational Molecular Biology and Algorithmic Approach, The MIT Press, Cambridge Mass.; Durbin et al. (1998) Biological Sequence Analysis: Probabilistic Models of Proteins and Nucleic Acids, Cambridge University Press, Cambridge, UK; Rashidi and Buehler (2000) Bioinformatic Basics: Applications in Biological Science and Medicine, CRC Press LLC, Boca Raton, Fla.; and Mount (2001) Bioinformatics: Sequence and Genome Analysis, Cold Spring Harbor Press, New York.

In one embodiment of the method, the frequency of each codon in each gene (or in each open reading frame (ORF)) is determined. The embodiment comprises identification of genes that have over- or under-represented codons. For each column of a matrix (see, above), i.e., for each codon, the genes are sorted according to the number of codons (sorted number list, SNL) or by frequency of codons (sorted frequency list, SFL). Next, a percentile threshold of significance is selected for each codon number and codon frequency. Other statistical tests at this decision point can optionally be considered. Each gene is reexamined and for each codon profile the gene is included if it falls above (or below) the frequency threshold, and discarded otherwise. See, below. The frequency threshold can be established by, e.g., examining the distribution curve of codon frequency for all genes, and setting a threshold based on metrics such as, e.g., standard deviation. Alternatively, one can examine the codon frequency of genes in a “training set” composed of genes or ORFs from bone fide PAIs extracted for the genome of the target organism (e.g., the organism), and set a codon frequency threshold based on the character of the genes in such a set. Thus, the lists are then truncated according to the significance thresholds to yield an Extreme Number List, ENL, from the SNL or an Extreme Frequency List, EFL, from the SFL. Such method thereby produces 64 lists of genes with extreme frequencies (i.e., one list for each codon). It will be appreciated that genes can occur on multiple lists, or on no list. Also, the threshold for inclusion of a particular codon on the extreme frequency list can be set differently for each codon.

Next in the analysis of the one or more genes to determine a distribution of at least one member of over/under represented codons is identification of codons for which possible genes of interest are included in the list of significant genes. Subsets of genes with relevant biological activity are chosen as genes of interest (GOIs). Next, the distribution of the GOIs is examined in the ENL and EFL of each codon. A codon is identified as a codon of interest (COI) when: a) a large number of GOIs occur in the ENL and/or the EFL of that codon relative to the overall number of genes in the set, or b) a gene known to be essential for or greatly contributory to, a phenotype of interest (e.g., such as virulence or survival, etc.) occurs in the ENL and/or EFL of that codon. Next, genes that may be of interest due to occurrence in the ENL and/or EFL of one more codon are identified. The distribution of genes in the ENL and the EFL of each codon is examined. Possible genes of interest (PGOIs) are identified for each codon. PGOIs may include open reading frames (ORFs) of undeterrmined function, ORFs with homology to GOIs, or groups of ORFs with known function that participate in a common biological pathway or related biological pathway and are identified in the ENL and/or EFL of a particular codon. Finally, the biological activity and/or regulation of each PGOI is experimental determined.

Next, at least one tRNA molecule responsible for encoding the at least one member of the pool of over- or under-represented codons is optionally identified. Optionally, the tRNAs are responsible for the translation of the codon of interest (COI) in the organisms of interest (OOI). The first step in this process is to identify the complete set of tRNAs (for example, by tRNAscan-SE program or other means well known to those of skill in the art) in the OOI. tRNAs of interest (TOI), e.g., those responsible for translating the COI, can be identified using knowledge of wobble rules and cognate codon-anticodon interactions. The TOI will represent the set of tRNAs whose characteristics (i.e. modifications) can be targeted by drugs to be developed that inhibit tRNA function.

Additionally, one or more tRNA characteristics essential for full tRNA function (including, but not limited to, biochemical modifications, tRNA synthetase identity-determinants, gene transcription promoter elements, and gene dosage, etc.) are optionally identified for each TOI. For example, biochemical modifications may be identified by hydrolysis of isolated TOIs followed by HPLC and mass spectroscopy analysis of modified bases. Thus, each characteristic or process identified optionally represents a possible drug target.

Furthermore, the present invention provides novel methods of gene regulation in bacterial organisms through the modulation of anticodons (transfer RNA) required for the synthesis of particular proteins and sets of proteins. As described herein, the distribution of codons within the gene complements of an organism is non-random. The occurrence of particular codons in certain genes is prominent. Additionally, sometimes functional families of genes are marked by the unusual frequency of a particular codon (see, below). For example, many pathogenicity genes of E. coli O157 are rich in the isoleucyl codon, ATA (AUA in the mRNA image of the gene). It is known that translation of this codon depends on a modification of cytosine at position 34 in the anticodon of nominal methionyl tRNA to lysidine. Therefore, expression of E. coli O157 virulence is likely to be strongly effected by drugs that interfere with the lysidine modification, thus modulating the virulence phenotype. See, below. Conversely, increasing the activity of the tRNAs responsible for translation of the ATA codon should enhance translation of the ATA-rich genes. This in effect is the path taken by nature. Non-pathogenic E. coli have one functional copy of the gene ileX, the tRNA gene for the lysinylated tRNA. In contrast, in E. coli O157, which contains the ATA enriched virulence genes, the number of ileX-like genes has increased to seven.

In some embodiments herein, the invention comprises identification of putative virulence genes (and/or genes affecting virulence) in organisms (e.g., typically pathogenic bacteria). The identification of such virulence genes occurs through determination of nucleic acid areas in the organism comprising, e.g., increased localization of rare codon usage (as compared, e.g., to the rest of the organism's genome). In other embodiments of the invention, genes that have been identified as putative virulence genes, etc. are optionally tested/screened for their possible effect/interaction on virulence of the organism. For example, such identified genes are optionally screened for virulence involvement through any known method of screening known to those of skill in the art (e.g., anti-sense screening, sense-suppression screening, homologous knockouts or recombinations, introduction of the putative gene into a non-virulent strain of the organism, introduction of the putative gene into a virulent strain of the organism (e.g., under a controllable promoter, thus allowing inducible expression to check for, e.g., enhancement of virulence and the like), etc.). In other embodiments of the invention, a virulence gene (e.g., one that has been identified through the methods herein via concentration of rare codon usage and/or one that has then been screened for actual impact on virulence) is optionally screened to identify and/or isolate one or more modulator/inhibitor of such virulence gene. Again, screens for such modulators are well known to those in the art (e.g., high throughput screening of such things as commercial/public libraries of peptides, nucleic acids, chemical entities, etc. though use of e.g., microtiter plates, robots, microfluidics, etc.). In yet other embodiments herein, any modulator/inhibitor of an identified virulence gene (or gene product depending upon context herein) is optionally used as a prophylactic and/or therapeutic agent to treat a subject against a virulent/pathogenic organism comprising the identified virulence gene/gene product.

Pathogenicity and Virulence

Pathogenicity islands and plasmids have been established as key elements that convey virulence and pathogenicity to a wide variety of bacteria. The genes in these elements have originated from a diverse array of species and most likely have been acquired by horizontal transfer. As such, the codon usage of the contained virulence genes in such plasmids and islands is characteristically divergent from that of the host genome. To measure this difference, the Codon Divergence Index (CDI), see, below, and the Rare Codon Divergence Index (RCDI), see, below, are developed herein. Furthermore, the current invention utilizes the fact that the increased presence of typically rare codons in many virulence genes can optionally leave these genes more susceptible to errors in translation, e.g., by tRNAs deficient in modifications of the anticodon loop.

Pathogenicity Elements

The number of pathogenicity elements discovered so far is remarkable. For example, large virulence plasmids have been identified in, e.g., EHEC (enterohemorrhagic) E. coli O157:H7 (pO157) (see, e.g., Burland, V. et al., (1998) Nuc Acids Res 26:4196-4204), EPEC (enteropathogenic) E. coli B171 (pB171) (see, e.g., Bach, S. et al., (2000) FEMS Microbiology Letters 183:289-294), S. typhi (R27 Resistance plasmid) (see, e.g., Sherburne, C., et al. (2000) Nuc Acids Res 28:2177-2186), three species of Yersinia, Shigella flexneri (pMYSH6000) (see, e.g., Andrews, G. et al. (1992) Infection and Immunity 60:3287-3295), and Bacillus anthracis (pXO1, pXO2) among others.

Additionally, phages encoding virulence elements have been discovered in E. coli and Shigella (e.g., Bacteriophages 933W, VT2-Sa, H-19B) (see, e.g., Hacker, J. et al., (1999) “Pathogenicity Islands and Other Mobile Virulence Elements” American Society for Microbiology, Washington, D.C. pp. 1-11) and Plunkett, G. et al., (1999) J Bacteriol 181(6):1767-78) as well as in Vibrio cholera (see, e.g., Karaolis, S. (1999) Nature 399(6734):375-9). Furthermore, the acquisition of pathogenicity islands appears to be among the major factors that have given rise to the separate lineages of virulent E. coli, Shigella, and Salmonella (see, e.g., Hacker J., et al., (1999) “Pathogenicity Islands and Other Mobile Virulence Elements” American Society for Microbiology, Washington D.C. pp. 35-58, 127-150 and 151-165). At least 12 PAIs have been identified in 8 E. coli serovars and 3 in Shigella serovars. At least 5 PAIs have helped to differentiate Salmonella species. Although pathogenicity islands and other pathogenicity elements have been most commonly identified in gram-negative enterobacteria, they have also been discovered in Listeria, several Bacilli species, Clostridia, Staphylococci and Streptococci. Furthermore, other genomic islands have been found, including a 500 kb symbiosis island of mesorhizobia (see, e.g., Sullivan, J., et al., (1998) Proc Natl Acad Sci USA 95(9):5145-9). Remarkably, this island is still mobile and is capable of transfer between mesorhizobia species in field and lab environments. It should thus be appreciated that large mobile genomic islands, including pathogenicity islands are found in diverse organisms.

Several pathogenicity elements are conserved between species. For example, probes specific to multiple regions of the LEE pathogenicity island (which encode, e.g., genes involved in gut wall attachment) hybridize to colonies of 8 serogroups of EPEC, 2 serotypes of EHEC, RDEC-1, Citrobacter freundii, and Hafnia alvei. See, e.g., McDaniel, T., et al., (1995) Proc Natl Acad Sci USA 92:1664-1668. In some embodiments, a serogroup comprises an inclusive collection of related “serotypes.” Thus, a serotype is optionally defined by consistent reactivity to a panel of, e.g., monoclonal antibodies, whereas a “serogroup” optionally shares reactivity to a panel of monoclonal antibodies. However, all member of the serogroup optionally may not react with all monoclonal antibodies. Additionally, the high-pathogenicity island (HPI) first isolated in Yersinia has recently been identified in 20 serotypes of E. coli, one serotype of Citrobacter diversus, and five species of Klebsiella. See, e.g., Back, S., (2000) supra, and Karch, H., et al., (1999) Infection and Immunity 67:5994-6001.

Codon Usage in Pathogenicity Islands and Other Mobile Virulence Elements

Aberrant nucleotide composition and codon usage

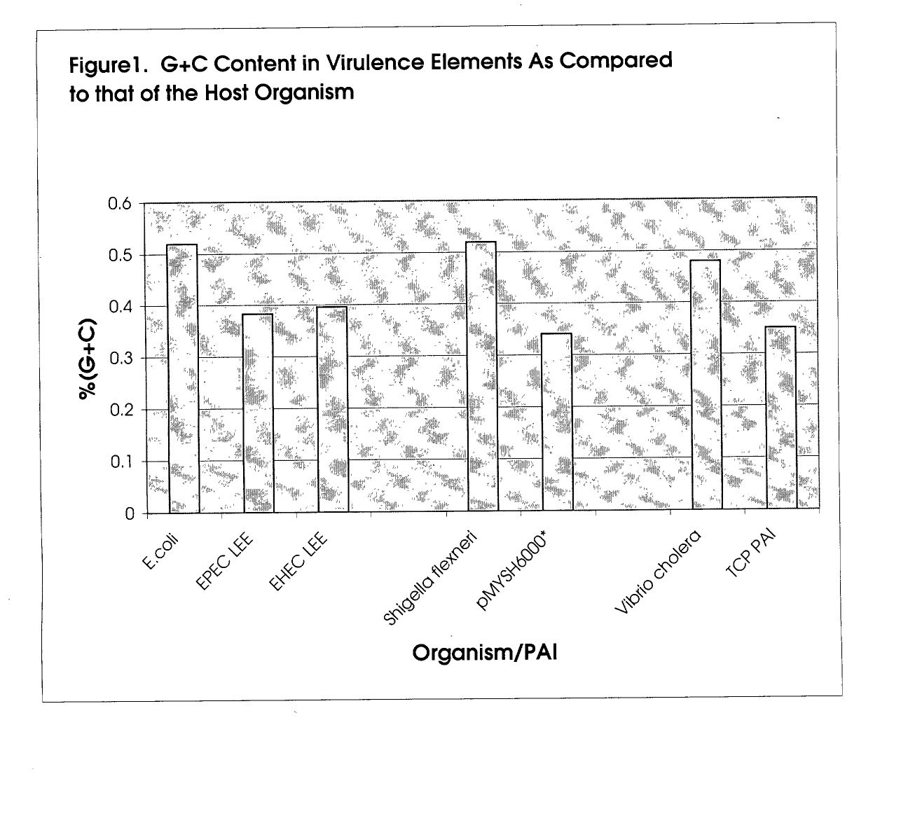

Since pathogenicity islands and other such elements are acquired by horizontal transfer, the G+C content of such elements oftentimes differs dramatically from that of the host organism. See, e.g., FIG. 1. The LEE pathogenicity islands of EPEC and EHEC have G+C contents of 38.3% and 39.59% as compared to 52% for the main E. coli chromosome. See, e.g., Perna, N. et al., (1998) Infection and Immunity 66:3810-3817 and Elliott, S., et al., (1998) Mol Microbiol 28(1):1-4. Also, the TCP pathogenicity island of Vibrio cholera has a G+C content of 35% while the rest of the V. cholera chromosome averages 48%. See, e.g., Hacker, et al., (1999) “Pathogenicity Islands and Other Mobile Virulence Elements” American Society for Microbiology, Washington D.C. pp. 167-187 and Karaolis, D., et al., (1998) Proc Natl Acad Sci USA 95(6):3134-9. Since many pathogenicity islands are themselves mosaic genetic structures derived from numerous sources, the G+C content oftentimes varies greatly even within a single element. Thus, a stretch of 35 genes in the pMYSH6000 has a G+C content of 34.1% as compared to approximately 52% for the Shigella chromosome and other parts of the plasmid. See, e.g., Hacker et al., (1999), supra, pp. 151-165. Due to the aberrant G+C contents of pathogenicity elements relative to their host genome, the codon usage of virulence genes may vary dramatically as compared to that of the rest of the genome. As explained herein, such differences optionally are utilized in the current invention to help target virulence sequences to reduce and/or eliminate virulence/pathogenicity of the organism.

As described above, pathogenicity islands and plasmids have been established as key elements that convey virulence and pathogenicity to a wide variety of bacteria. The genes in these elements have originated from a diverse array of species and have been acquired by horizontal transfer. As such, the codon usage of the contained virulence genes is characteristically divergent from that of the host genome. To measure this difference, the Codon Divergence Index (CDI) and the Rare Codon Divergence Index (RCDI) were developed in the current invention. Furthermore, as described above, the current invention utilizes the fact that the increased presence of typically rare codons in many virulence genes may leave these genes more susceptible to errors in translation, e.g., by tRNAs deficient in modifications of the anticodon loop, etc. and thus to control/modulate.

tRNA

tRNA modifications have been demonstrated to play several key roles in maintaining the tRNA's ability to faithfully decode an mRNA sequence. See, e.g., Qian, Q., et al., (1998) J Bacteriol 180(7):1808-13; Grosjean, H., et al., (1995) Biochimie 77:3-6; Esberg, B., et al., (1995) J Bacteriol 177(8):1967-75; and Grosjean, H., et al., (1998) “Modification and Editing of RNA,” American Society for Microbiology, Washington D.C., pp. 493-516. Furthermore, tRNA modifications have been implicated in full and proper translation of virulence genes in Shigella flexneri (see, e.g., Durand, J., et al., (1994) J Bacteriol 176(15):4627-34; Durand, J., et al., (1997) J Bacteriol 179(18):5777-82; and Durand, J., et al., (2000) Mol Microbiol 35(4):924-35) and in the plant pathogen Agrobacterium tumefaciens (see, e.g., Gray, J., et al. (1992) J Bacteriol 174(4):1086-9823). Modifications found at position 34 of the anticodon have been shown to change the coding capabilities of a particular tRNA by expanding or restricting the wobble rules at that position. For example, queosine (Q) replaces a guanosine at position 34 in Tyr, His, Asp, and Asn tRNAs and helps prevent misreading of the TAA/TAG STOP codons, and may prevent misreading of Gln, Lys, and Glu codons by restricting wobble. Alternatively, the lysidine modification at position 34 of the rare bacterial ileX tRNA changes its coding capacities from AUG to AUA (see, e.g., Muramatsu, T., et al.,(1988) Nature 336(6195):179-81). Furthermore, modifications adjacent to the anticodon at position 37, including i6A and t6A, have been demonstrated to effect strand slipping and stop codon read through (see, e.g., Qian, Q., et al., (1998) J Bacteriol 180(7):1808-13; Esberg, B., et al., (1995) J Bacteriol 177(8):1967-75; and Miller, J., et al., (1976) Nuc Acids Res 3(5): 1185-201) and effects the fidelity of codon/anticodon interactions.

Attempted overexpression of heterologous proteins containing high levels of rare codons in E. coli has led to poor translation and even growth inhibition. Such effects were reversed when the appropriate cognate tRNA was also overexpressed. See, e.g., Del Tito, B., et al., (1995) J Bacteriol 177(24):7086-91 and Zahn, K., (1996) J Bacteriol 178(10):2926-33. Such effect may occur because the level of any specific tRNA in a cell is correlated to the frequency of codon usage for the codon it recognizes. See, e.g., Kanaya, S., et al., (1999) Gene 238(1):143-55. If a gene contains a particular codon at a frequency that far exceeds the average use in the genome, problems in translation may be expected due to the relative scarcity of that tRNA in the cell. Overexpression of heterologous genes with codon usage more in line with that of the host's codon usage occurs without problems in translation or growth. While the above studies deal with the overexpression of proteins, they indicate that the levels of cognate tRNAs of rare codons may limit translation of genes which contain a high level of that codon. Furthermore, evidence exists that the rare arginine tRNA modulates expression of the int lambda phage gene in vivo, an effect that may be dependent on increased use of the rare AGA and AGG arginine codons in the int gene. See, e.g., Zahn, K., et al., (1996) Mol Microbiol 21(1):698-76.

The methods of the present invention can be used to identify a number of targets of bacterial origin, including, but not limited to, tRNA molecules involved in, for example, the expression of a virulence phenotype, expression of a developmental phenotype, or expression of a environmental response phenotype. See, below. Furthermore, the methods of the present invention optionally further comprise the step of designing one or more synthetic genes to conform in codon use to particular gene subset (for example, a new virulence gene). The methods of the present invention can also be used to define gene subsets (by membership on a particular extreme frequency codon list); these gene subsets are then usable as inputs for subsequent experimental procedures. Additionally, the methods can be employed to design systems in which expression of gene subsets can be modulated by “gene dosage”, i.e. by adding to or subtracting from the number of appropriate tRNA genes.

PAIs and other mobile virulence elements have been examined, focusing on their distribution and codon usage (specifically distribution of rare codons) as compared to the host. Pathogenicity elements are shown to be more susceptible than host genes to fluxes in the functional pool of certain tRNAs due to their increased level of rare codons. To identify pathogenicity elements with codon usage divergent from that of the host, the CDI and RCDI methods were developed herein and calculated for genes of known pathogenicity elements. Specific codons are identified that have an increased use in pathogenicity elements while their cognate tRNAs and modifications of their cognate tRNAs are cited. Such cognate tRNAs, if inhibited, may lead to an increased rate of misincorporation and termination during translation of pathogenicity genes due to the increased use of rare codons in these elements.

Codon Divergence Index and Rare Codon Divergence Index

In order to characterize the codon bias in, e.g., pathogenicity elements and the like, the Codon Divergence Index (CDI) and the Rare Codon Divergence Index (RCDI) are used herein. These indices measure the average difference in codon usage between a gene and a reference genome for all codons (CDI) and for the 10 rarest codons (RCDI). Other indices, such as the Codon Adaptivity Index (CAI), which have previously been developed (see, e.g., Kanaya, S., et al., (1999) Gene 238(1):143-55), take into account the amino acid bias of the gene and are used to help establish genetic distances, to determine horizontal transfer into the genome, or to determine which codons are favored within a family box in different gene classes. Here, however, the concern is strictly with the frequency of codon usage and does not take into account the amino acid bias of the gene. Therefore, such previously developed measurements (e.g., the CAI) are not used herein.

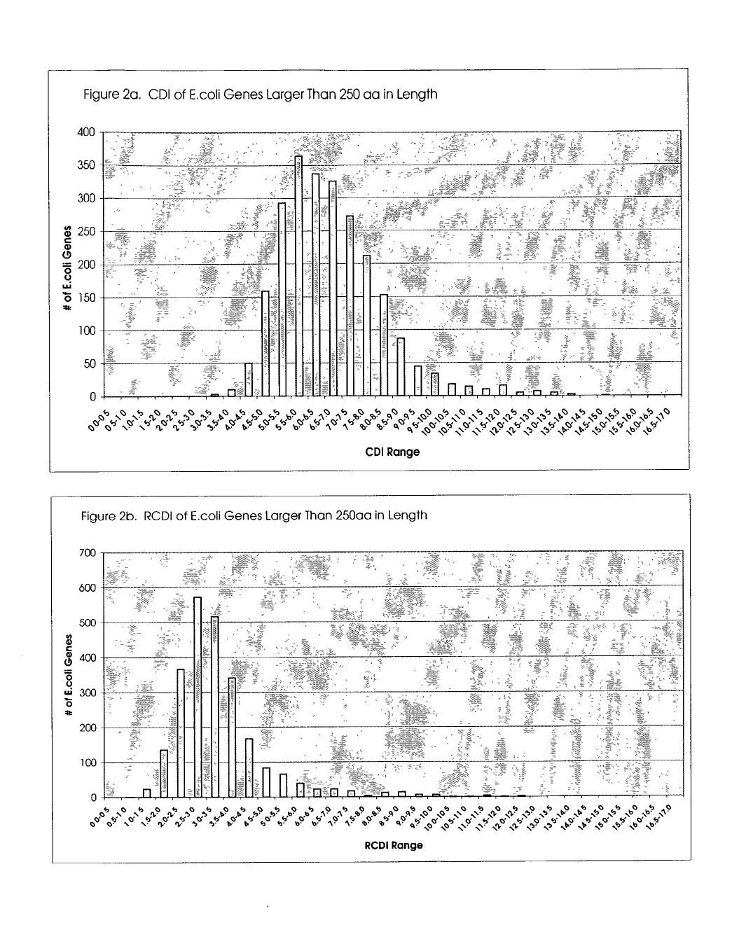

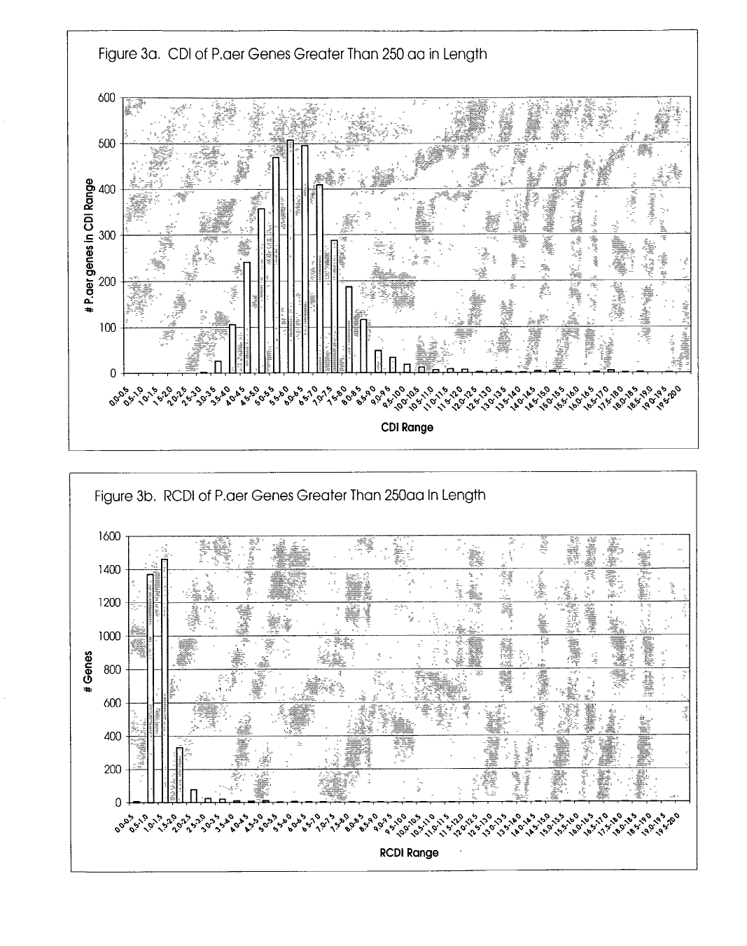

To illustrate these concepts, CDIs and RCDIs were calculated (see, below) for each gene in the host genomes of E. coli and P. aeruginosa. Genes smaller than 250 amino acids in length were excluded from further analysis since it was determined that genes with less than 250 codons have skewed codon frequencies, and therefore skewed CDIs and RCDIs, due to limited codon representation. Distributions of CDI and RCDI scores are shown in FIG. 2 and FIG. 3 for E. coli and P. aeruginosa respectively. The average CDI scores for E. coli and P. aeruginosa genes are 6.71 and 6.13 respectively, indicating that the average difference in codon usage per 1000 codons for any given codon is about 6.5 for both E. coli and P. aeruginosa. RCDI scores, indicate that the frequency of codon usage for rare codons varies less than for more common codons, especially in P. aeruginosa. The average RCDI in E. coli was 3.38 whereas the average RCDI in P. aeruginosa was 1.30. Only 5% of the genes in P. aeruginosa had RCDI scores greater than 2, indicating that the rarest 10 codons in P. aeruginosa genes are rare in nearly every gene.

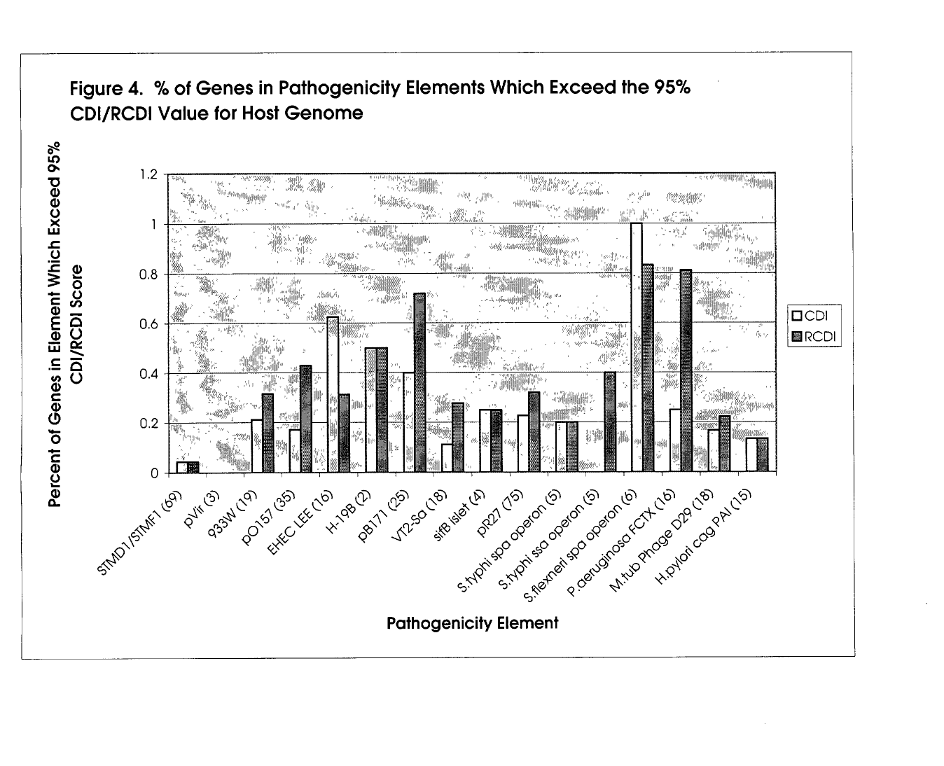

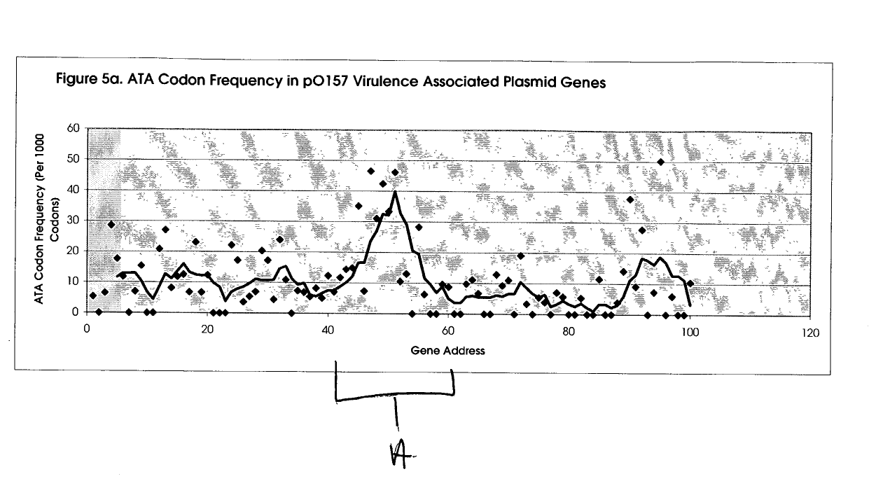

Scores were determined for which 95% of the scores of genes in the reference genome fell below (see, Table 1a). For instance, in E. coli, 95% of the genes greater than 250 amino acids in length had CDI scores below 9.40 and RCDI scores below 5.87. Thus, only 121 of the 2427 genes greater than 250 amino acids in E. coli have CDI scores greater than 9.40. CDIs and RCDIs were then calculated for the genes in two stretches of the Salmonella chromosome, STMD1 and STMF1, which have been released by the Salmonella typhi sequencing project. E. coli K12 was used as the reference genome. Of the 69 genes greater than 250 amino acids in the two S. typhi control regions, only 3 (4.4%) have scores that exceeded the 95% scores for the CDI and another 3 have scores that exceed the 95% score of the RCDI (see, FIG. 4 and Table 1c). E. coli K12 was also used as the reference organism for Shigella since both genomes have very similar G+C contents (52%) (see, e.g., Groisman, E., et al., (1993) EMBO J 12:3779-3787). Genes that have been sequenced in both genomes have nucleotide sequences that are very similar to each other. Evidence exists that the two bacteria are actually different serotypes of the same species (see, e.g., Hacker, J., et al., (1999), supra, pp. 151-156) Thus, using codon usages from E. coli K12 in evaluating Salmonella and Shigella genes are valid comparisons.

Table 1 gives a comprehensive list of pathogenicity genes examined for which their CDI or RCDI is greater than the 95% threshold score in the reference genome. Every pathogenicity element analyzed, except pVir from Campylobacter jejuni, was found to be enriched in genes that exceed this threshold, indicating that the genes in these genetic regions have codon usage that is very divergent from that of the host organism. If codon usage was similar in these elements as compared to the host genome, one would expect only 5% of the genes to have scores above the 95% threshold for the CDI and RCDI. However, 6 out of the 19 genes greater than 250 amino acids in length (31.6%) in the Shiga-toxin 2 converting bacteriophage 933W have an RCDI score above the 95% threshold while 15 of 35 (42.9%) genes of the EHEC large pathogenicity plasmid, pO157 and 10 of 16 (62.5%) genes in the EHEC LEE PAI have scores above the 95% threshold of the CDI. In the cytotoxin converting phage of P. aeruginosa, ΦCTX, 13 of 16 (81.3%) genes have RCDI values above the 95% threshold for that genome. See also, FIG. 4.

Interestingly, of all the genes in pathogenicity islands, it is often the virulence factors essential for virulence that have the highest CDI and RCDI values. The serine-threonine kinase and shiga-toxin 2 gene of the 933W bacteriophage have the highest RCDI scores in this element while the 3 hemolysin genes, along with a 3170 amino acid putative cytotoxin, account for the 4 genes in pO157 with the highest CDI. Similarly virF, the most upstream regulator of virulence in Shigella, has among the largest CDI and RCDI scores of the sequences from E. coli, Shigella, and Salmonella that were analyzed. Indeed, there is only 1 gene in the E. coli K12 genome (appY) which has an RCDI greater than that of virF (12.41 versus 12.11).

Although mobile virulence elements have not been identified in Mycobacterium tuberculosis, it can be seen from analysis of the Mycobacteriophage D29 that the genes of phages of this bacterium may also have codon usages which differ dramatically from that of the host. See, Table 1c.

TABLE 1a
Percentile scores for host genomes.

| Percentile | # E. coli Genes | CDI Value | RCDI Value |
|---|---|---|---|
| 98% | 49 | 10.94 | 7.93 |
| 95% | 121 | 9.40 | 5.87 |
| 92% | 194 | 8.86 | 5.18 |
| 90% | 243 | 8.54 | 4.78 |
| Percentile | # P. aer. Genes | CDI Value | RCDI Value |
| 98% | 68 | 9.79 | 3.82 |
| 95% | 169 | 8.42 | 2.09 |
| 92% | 269 | 8.02 | 1.85 |
| 90% | 337 | 7.81 | 1.70 |
| Percentile | # M. tub. Genes | CDI Value | RCDI Value |
| 98% | 46 | 10.57 | 4.87 |
| 95% | 116 | 8.77 | 4.17 |
| 92% | 185 | 8.13 | 3.84 |
| 90% | 230 | 7.75 | 3.66 |

TABLE 1b
CDI and RCDI scores of virulence-associated genes.

| Mobile | | | | |
|---|---|---|---|---|
| Pathogenicity | | | | |
| Element [# | Gene | | | |
| Genes > 250aa] | Name | Function | CDI* | RCDI |
| 933W [19] | stk | serine-threonine kinase | 11.74 | 11.98 |
| | L0121 | Putative tail fiber | 10.51 | 5.9 |
| | stx2A | Shiga-like toxin 2 | 9.80 | 8.10 |
| | | subunit A | | |
| | L0095 | Hypothetical protein | 9.57 | 6.94 |
| | L0112 | Putative terminase small | 8.62 | 6.52 |
| | | subunit | | |
| | int | Phage integrase | 7.10 | 7.10 |
| pO157 [35] | L7095 | putative cytotoxin | 11.91 | 8.93 |
| | EHEC- | hemolysin transport | 11.40 | 8.78 |
| | hlyD | | | |
| | EHEC- | hemolysin toxin protein | 11.38 | 5.88 |
| | hlyA | | | |
| | EHEC- | hemolysin transport | 10.80 | 8.52 |
| | hlyB | | | |
| | L7026 | hypothetical protein | 10.31 | 7.03 |
| | etpN | type II secretion protein | 9.63 | 7.37 |
| | L7072 | hypothetical protein | 9.03 | 8.64 |
| | redF | resolvase | 8.65 | 6.78 |
| | L7046 | ORFB of IS911 | 8.58 | 7.19 |
| | etpK | type II secretion protein | 8.05 | 7.54 |
| | L7029 | putative acyltransferase | 7.49 | 7.47 |
| | L7076 | hypothetical protein | 8.31 | 7.05 |
| | traI | DNA helicase | 8.22 | 6.94 |
| | L7056 | replication protein | 8.14 | 6.58 |
| | L7027 | hypothetical protein | 6.29 | 6.07 |
| LEE | L0016 | Unknown | 14.53 | 9.30 |
| (O157:H7) [16] | | | | |
| | escT | Type III secretion apparatus | 12.56 | 11.68 |
| | L0023 | Unknown | 11.60 | 3.61 |
| | escU | Type III secretion apparatus | 11.46 | 7.32 |
| | L0056 | Similar to E. coli YijK | 10.33 | 8.71 |
| | escC | Type III secretion apparatus | 10.14 | 4.72 |
| | sepQ | Type III secretion apparatus | 10.14 | 5.83 |
| | espB | Secreted protein | 10.04 | 3.83 |
| | L0055 | Similar to Shigella VirA | 9.47 | 5.87 |
| | tir | Translocated intimin | 9.46 | 4.75 |
| | | receptor | | |
| H-19B [2] | slt-IA | Shiga-like toxin1 subunit A | 11.66 | 7.25 |
| pB171 [25] | bfpT | Transcriptional regulator of | 13.84 | 10.66 |
| | | bfpA-L operon | | |
| | orf35 | Homologous to C-terminus | 13.18 | 10.51 |
| | | of EHEC ToxB | | |
| | bfpP | prepilin peptidase | 13.09 | 11.86 |
| | orf62 | putative amino acid | 11.84 | 6.40 |
| | | antiporter | | |
| | orf63 | putative glutamate | 11.77 | 10.39 |
| | | decarboxylase | | |
| | bfpE | biosynthesis of bundle- | 10.16 | 9.66 |
| | | forming pili | | |
| | bfpD | biosynthesis of bundle- | 10.01 | 7.42 |
| | | forming pili | | |
| | bfpB | biosynthesis of bundle- | 9.65 | 3.89 |
| | | forming pili | | |
| | bfpF | biosynthesis of bundle- | 9.65 | 4.58 |
| | | forming pili | | |
| | bfpC | biosynthesis of bundle- | 9.48 | 7.46 |
| | | forming pili | | |
| | orf55 | Transposase | 8.97 | 11.23 |
| | rsvB | similar to resolvase or F | 8.94 | 7.54 |
| | | plasmid | | |
| | orf22 | Transposase | 7.71 | 9.25 |
| | orf34 | Transposase | 7.71 | 9.25 |
| | orf59 | reverse transcriptase-like | 6.51 | 7.58 |
| | | protein | | |
| | piv | pilin gene inverting protein | 7.33 | 7.14 |
| | orf31 | similar to EHEC L0015 | 6.74 | 6.50 |
| | | hypothetical protein | | |
| | orf51 | similar to EHEC L0015 | 6.66 | 6.50 |
| | | hypothetical protein | | |
| | orf41 | Transposase | 7.62 | 6.44 |
| | ofr77 | Transposase | 7.34 | 6.15 |
| VT2-Sa [18] | | hypothetical protein | 10.51 | 5.91 |
| | stx2A | Shiga toxin 2 subunit A | 9.80 | 8.10 |
| | | hypothetical protein | 8.62 | 6.52 |
| | int | integrase | 7.03 | 7.10 |
| | O | O-protein | 8.26 | 6.06 |
| S. entero sifB | sifB | Virulence factor; involved in | 10.47 | 10.76 |
| islet [4] | | systemic disease | | |
| S. typhi pR27 | R0151 | hypothetical protein | 12.15 | 7.11 |
| [75] | gltS | putative glutamate permease | 11.09 | 4.95 |
| | R0002 | hypothetical protein | 10.83 | 2.69 |
| | R0149 | hypothetical protein | 10.68 | 7.76 |
| | R0037 | hypothetical protein | 10.67 | 3.97 |
| | R0201 | hypothetical protein | 10.18 | 5.87 |
| | tetA | tetracycline antiporter protein | 10.08 | 3.64 |
| | R0123 | hypothetical protein | 10.07 | 4.05 |
| | R0199 | hypothetical protein | 9.96 | 6.66 |
| | R0019 | putative partition protein | 9.93 | 5.81 |
| | R0031 | hypothetical protein | 9.59 | 5.54 |
| | trhV | putative transfer protein | 9.59 | 4.27 |
| | R0085 | IS10 transposase | 9.58 | 8.17 |
| | R0202 | hypothetical protein | 9.58 | 7.70 |
| | R0076 | putative IS10 transposase | 9.57 | 7.83 |
| | R0004 | hypothetical protein | 9.51 | 3.51 |
| | trhE | hypothetical protein | 9.50 | 5.32 |
| | htdT | putative transfer protein | 9.28 | 7.84 |
| | R0016 | hypothetical protein | 9.16 | 6.82 |
| | R0046 | IS30 transposase | 9.07 | 11.48 |
| | R0200 | hypothetical protein | 8.94 | 6.19 |
| | trhU | putative sex pilus assembly | 8.40 | 9.16 |
| | | and synthesis | | |
| | mucB | putative UV protection protein | 6.79 | 7.49 |
| | R0195 | IS2 hypothetical orf | 7.60 | 7.36 |
| | trhI | utative DNA helicase | 7.67 | 7.00 |
| | R0204 | hypothetical protein | 8.00 | 6.74 |
| | R0175 | hypothetical protein | 7.47 | 6.72 |
| | R0186 | hypothetical protein | 7.07 | 6.52 |
| | R0041 | putative DNA adenine | 7.54 | 6.50 |
| | | methylase (dam) | | |
| | R0152 | hypothetical protein | 8.13 | 6.44 |
| | R0156 | hypothetical protein | 6.93 | 6.37 |
| | R0168 | hypothetical protein | 6.14 | 6.35 |
| | R0015 | hypothetical protein | 8.35 | 6.21 |
| | R0040 | hypothetical protein | 8.32 | 5.90 |
| S. typhi spa | spaL | surface presentation of | 10.46 | 13.59 |
| operon (Part of | | antigens; type III secretion | | |
| SPI-1 PAI) | | (essential for entry into | | |
| [5] | | epithelial cells) | | |
| S. typhi ssa | ssaL | typeIII secretion apparatus | 9.21 | 6.83 |
| operon (Part of | | | | |
| SPI-2 PAI) | | | | |
| [5] | ssaN | typeIII secretion apparatus | 7.23 | 9.14 |
| S. flexneri spa | spa29 | Type III secretion apparatus; | 14.16 | 7.65 |
| operon | | necessary for epithelial cell | | |
| (Part of | | invasion | | |
| pMYSH6000 | | | | |
| virulence | | | | |
| plasmid) [6] | | | | |
| | spa32 | Type III secretion apparatus; | 12.91 | 7.26 |
| | | necessary for epithelial cell | | |
| | | invasion; homologous to S. | | |
| | | typhi spaN | | |
| | spa33 | Type III secretion apparatus; | 12.31 | 4.94 |
| | | necessary for epithelial cell | | |
| | | invasion; homologous to S. | | |
| | | typhi spaO | | |
| | mxiA | export of Shigella virulence | 12.22 | 9.31 |
| | | factors; essential for invasion | | |
| | | of HeLa cells | | |
| | spa40 | Type III secretion apparatus; | 12.10 | 8.78 |
| | | necessary for epithelial cell | | |
| | | invasion | | |
| | spa47 | Type III secretion apparatus; | 11.50 | 8.53 |
| | | necessary for epithelial cell | | |
| | | invasion; homologous to S. | | |
| | | typhi spaL | | |
| S. flex. | virF | Primary virulence regulatory | 13.64 | 12.11 |
| unmapped viru- | | protein | | |
| lence elements | | | | |
| [1] | | | | |
| P. aeruginosa | ORF15 | Hypothetical protein | 17.34 | 12.40 |
| φCTX [16] | | | | |
| | ORF28 | Hypothetical protein | 16.77 | 10.71 |
| | ctx | cytotoxin | 11.18 | 2.91 |
| | H | tail synthesis | 8.47 | 3.46 |
| | int | integrase | 7.39 | 6.35 |
| | ORF11 | Hypothetical protein | 6.93 | 3.84 |
| | Q | capsid synthesis | 6.60 | 3.01 |
| | J | tail synthesis | 7.41 | 2.95 |
| | T | tail synthesis | 5.72 | 2.78 |
| | N | capsid synthesis | 6.55 | 2.57 |
| | D | tail synthesis | 6.07 | 2.55 |
| | O | capsid synthesis | 6.82 | 2.18 |
| | ORF41 | Hypothetical protein | 4.81 | 2.10 |
| H. pylori cag | cagT | Secretion of virulence factors | 9.21 | 4.78 |
| PAI [15] | | (?) | | |
| | cagH | Secretion of virulence factors | 8.65 | 1.92 |
| | | (?) | | |
| | virB11_1 | Secretion of virulence factors | 6.82 | 4.38 |
| | | (?) | | |

*CDI and RCDI values that exceed the 98^{th} percentile score in the host genome are bold and underlined; values that exceed the 95^{th} percentile score are in bold; values that exceed the 92^{nd} percentile score are underlined; while values that exceed the 90^{th} percentile score are italicized.

TABLE 1c
CDI and RCDI scores of non-virulence-associated genes.

| Mobile | | | | |
|---|---|---|---|---|
| Pathogenicity | Gene Name | Function | CDI* | RCDI |
| S. typhi STMD1 | ilvC | S. typhimurium | 10.06 | 3.45 |
| [52] | | ketol-acid | | |
| | | reductoisomerase | | |
| | yigW | hypothetical protein | 7.04 | 7.30 |
| | yifB | hypothetical protein | 6.65 | 6.54 |
| S. typhi STMF1 | tufB | translation elongation | 12.39 | 3.73 |
| [17] | | factor TU (EF-TU) | | |
| | STMF1.17 | hypothetical protein | 11.92 | 7.43 |
| M. tub Phage | gp27 | minor tail subunit | 10.03 | 2.33 |
| D29 [18] | | | | |
| | gp54 | hypothetical protein | 9.04 | 2.32 |
| | gp17 | major head subunit | 8.88 | 2.65 |
| | gp2 | hypothetical protein | 8.73 | 4.18 |
| | gp12 | hypothetical protein | 8.64 | 5.02 |
| | gp15 | hypothetical protein | 8.11 | 4.61 |
| | gp10 | hypothetical protein | 6.76 | 4.91 |

*CDI and RCDI values that exceed the 98^{th} percentile score in the host genome are bold and underlined; values that exceed the 95^{th} percentile score are in bold; values that exceed the 92^{nd} percentile score are underlined; while values that exceed the 90^{th} percentile score are italicized.

Calculation of CDI and RCDI.

All DNA sequences used in the following examples were downloaded from GenBank. Accession numbers for these sequences are listed in Tables 2a-b and 3. The CDI and RCDI were calculated for each virulence loci as follows:

CDI: For each host genome, the frequency of each codon per 1000 codons (fᵢ) was calculated for the entire genome for all 61 non-stop codons: fᵢ=[(#codonᵢ)(1000 codons)/(#codons in all ORFs)] where i is the set from 1 to 61. For each ORF in the pathogenicity loci, the frequency of each codon per 1000 codons (cᵢ) was calculated for all 61 non-stop codons: cᵢ=[(#codonᵢ)(1000 codons)/(#codons ORF)] where i is the set from 1 to 61. The CDI for each gene was then calculated as the average absolute difference between cᵢ and fᵢ:

$$CDI = \frac{{\sum }_{i - 1}^{61} \vert {c}_{i} - {f}_{i} \vert}{61} \qquad ( Equation 1 )$$

RCDI: The RCDI was calculated as above for the CDI except that i is the set 1 to 10 of the 10 rarest codons in the host genome. The RCDI is then the average absolute difference between cᵢ and fᵢ for the 10 rarest codons.

$$RCDI = \frac{{\sum }_{i - 1}^{10} \vert {c}_{i} - {f}_{i} \vert}{10} \qquad ( Equation 2 )$$

Percentile scores were calculated for each host genome. 95 percentile scores were determined for the CDI and RCDI of each host genome at which point 95% of the genes greater than 250 codons had CDI and RCDI scores that were smaller. Genes smaller than 250 codons were not used because, as explained above, it was determined that the codon frequencies, and hence their CDI and RCDI scores, were skewed due to limited codon representation (data not shown).

To illustrate CDI and RCDI, several pathogenicity elements which have been identified and sequenced from a wide array of organisms, including several strains of pathogenic E. coli, Shigella, Salmonella, Vibrio cholera, Campylobacter jejuni, and Helicobacter pylori were examined. Many of these are organized in pathogenicity islands and an estimated 75% of virulence genes are organized in genetic structures flanked by tRNA genes (e.g., as seen via visual inspection of genome maps). Several of these pathogenicity elements were analyzed herein and are listed in Table 2a. Non-virulence associated genes that were analyzed for comparison are listed in Table 2b.

TABLE 2a
Analysis of Mobile Virulence Elements.

| | Pathogenicity | | |
|---|---|---|---|
| Organism(s) | Element | Function | Accession # |
| E. coli O157:H7 | LEE PAI | TypeIII Secretion, invasion | AF071034 |
| (EHEC) | | | |
| | Bacteriophage | Stx2 production | AF125520 |
| | 933W | | |
| | Bacteriophage | Stx2 production | AP000363 |
| | VT2-Sa | | |
| | pO157 plasmid | Hemolysin, toxin, type II secretion | AF074613 |
| E. coli B171 | pB171 plasmid | Type IV pili | AB024946 |
| (EPEC) | | | |
| E. coli/Shigella | Bacteriophage | Stx1 production | AF034975 |
| | H-19B | | |
| S. enteritidis | SifB | Similar to sifA pathogenicity islet in | AF128839 |
| | pathogenicity | S. typhi; required for systemic | |
| | islet | infection and lethality in mice | |
| S. typhi- | R27 large | Multi-drug resistance | AF250878 |
| | resistance | | |
| | plasmid | | |
| | spa operon of | TypeIII secretion apparatus; | X73525 |
| | Salmonella | necessary for invasion of host | |
| | Pathogenicity | epithelial cells | |
| | Island 1 | | |
| | (SPI-1) | | |
| | ssa operon of | Type III secretion apparatus; | Y09357 |
| | Salmonella | necessary for intracellular | |
| | Pathogenicity | proliferation, systemic disease | |
| | Island 2 | | |
| | (SPI-2) | | |
| S. flexneri | spa operon of | TypeIII secretion apparatus; | D13663 |
| | pMYSH6000 | necessary for invasion of host | |
| | | epithelial cells; homologous to spa | |
| | | operon of S. typhi | |
| | mxiA | export of Shigella virulence factors; | M91664 |
| | | essential for invasion of HeLa cells | |
| | virF | Primary virulence regulatory protein | X16661 |
| C. jejuni | pVir | Virulence-associated plasmid | AF226280 |
| H. pylori | cag PAI | Secretion of virulence determinants | AE000511 |
| P. aeruginosa | ΦCTX | Cytotoxin converting phage | AB008550 |

TABLE 2b
Analysis of Non-Virulence Associated Genes.

| Organism(s) | Sequence | Function | Accession # |
|---|---|---|---|
| M. tuberculosis | Mycobacteriophage | Unknown | NC_001900 |
| | D29 | | |
| S. typhi | STMD1 | Control Sequence | AF233324 |
| | STMF1 | Control Sequence | AF170176 |

TABLE 3
Effects of leuX and selC tRNAs on the virulence properties of
UPEC.

| | selC+/ | selC−/ | selC+/ | |
|---|---|---|---|---|
| | leuX+ | leuX− | leuX− | selC−/leuX+ |
| FDH production | + | − | + | − |
| Anaerobic Growth | + | − | + | − |
| Typ 1 Fimbria | + | − | − | + |
| Flagella | + | − | − | + |
| Motility | + | − | − | + |
| Iron uptake | + | − | − | + |
| Enterobactin production | + | − | − | + |
| Serum resistance | + | − | − | + |
| Survival in mouse bladder | + | − | − | + |
| Colonization of large intestine | + | − | − | + |
| (in competition w/wt) | | | | |
| In vivo virulence | + | − | − | + |

### FURTHER EXAMPLES OF CDI/RCDI CALCULATIONS IN SELECTING CODONS OF INTEREST

### Example 1.

Identification of a codon of interest (ATA) (see, Table 4a) of E. coli O157 by the method described above, and comparison to codons not identified as COI (ATG, CTG) (see, Tables 4c and 4d). The Extreme Frequency List (EFL) or Extreme Number List (ENL), see, Table 4, was generated as described above with 99th percentile codon frequencies (for EFL) or codon number (for ENL) used as the statistical threshold for each codon. GOIs were identified as described above. ATA was chosen as a COI due to its high percentage of GOIs in the EFL (13.0% of genes in AFL) as compared to other codons (3.7% in the EFL of ATG and 5.6% in the EFL of CTG).

TABLE 4a
EFL of ATA codon in E. coli O157.

| Frequency | | | |
|---|---|---|---|
| (#ATA/1000)* | | Description | GOI? |
| 46.51 | hlyC | hemolysin transport | GOI |
| 46.88 | stx2A | shiga-like toxin II A subunit encoded by bacteriophage BP- | GOI |
| | | 933W | |
| 47.17 | yehK | orf; Unknown function | |
| 47.62 | — | unknown protein encoded by prophage CP-933O | |
| 47.62 | — | orf; Unknown function | |
| 47.62 | — | orf; Unknown function | |
| 47.97 | — | unknown protein encoded by prophage CP-933N | |
| 48.71 | stk | putative serine/threonine kinase encoded by bacteriophage | GOI |
| | | BP-933W | |
| 49.18 | — | orf; Unknown function | |
| 50 | — | orf; Unknown function | |
| 50 | — | orf; Unknown function | |
| 50 | — | orf; Unknown function | |
| 50.16 | L7095 | putative cytotoxin | GOI |
| 50.46 | escR | type III secretion apparatus protein | GOI |
| 51.16 | — | orf; Unknown function | |
| 51.16 | — | orf; Unknown function | |
| 51.28 | — | orf; Unknown function | |
| 51.66 | — | putative regulator; Not classified | |
| 51.72 | — | orf; Unknown function | |
| 52.63 | — | orf; Unknown function | |
| 52.63 | — | orf; Unknown function | |
| 52.63 | — | unknown protein encoded within prophage CP-933R | |
| 53.19 | — | orf; Unknown function | |
| 53.19 | — | orf; Unknown function | |
| 53.57 | — | unknown protein encoded by prophage CP-933K | |
| 54.05 | — | unknown protein encoded by prophage CP-933Y | |
| 54.79 | — | orf; Unknown function | |
| 54.79 | — | orf; Unknown function | |
| 54.88 | — | orf; Unknown function | |
| 56.41 | — | unknown protein encoded by prophage CP-933N | |
| 57.47 | — | orf; Unknown function | |
| 58.56 | — | putative integral membrane protein-component of typeIII | |
| | | secretion apparatus | |
| 63.06 | wbdR | acetyl transferase; O-antigen biosynthesis | |
| 63.29 | — | orf; Unknown function | |
| 63.29 | wzy | O antigen polymerase | |
| 65.64 | escT | type III secretion apparatus protein | GOI |
| 66.67 | — | orf; Unknown function (Rhs Element Associated) | |
| 66.67 | — | orf; Unknown function | |
| 67.57 | — | rhsC protein in rhs element | |
| 75 | — | orf; Unknown function | |
| 75.27 | ybbV | orf, hypothetical protein | |
| 75.47 | — | unknown protein encoded by prophage CP-933N | |
| 76.92 | — | unknown protein encoded by prophage CP-933K | |
| 77.59 | wzx | O antigen flippase Wzx | |
| 78.43 | — | orf; Unknown function | |
| 80.46 | ybbD | orf, hypothetical protein | |
| 81.3 | — | orf; Unknown function | |
| 81.3 | — | orf; Unknown function | |
| 85.71 | — | orf; Unknown function | |
| 88.89 | escS | type III secretion apparatus protein | GOI |
| 91.74 | ybfB | orf, hypothetical protein | |
| 95.89 | — | orf; Unknown function | |
| 212.12 | — | orf; Unknown function | |
| 212.12 | — | orf; Unknown function | |
| 54 Genes | | | 7GOI |

13.0% GOI
*99 percentile ATA codon frequency used as statistical threshold.

TABLE 4b
ENL of ATA codon in E. coli O157.

| # ATA | | | |
|---|---|---|---|
| Codons* | Gene | Description | GOI? |
| 15 | — | orf; Unknown function | |
| 15 | stx2A | shiga-like toxin II A subunit encoded | GOI |
| | | by bacteriophage BP-933W | |
| 15 | — | unknown protein encoded by prophage | |
| | | CP-933N | |
| 15 | — | putative cytotoxin | GOI |
| 15 | waaL | putative LPS biosynthesis enzyme | |
| 16 | — | orf; Unknown function | |
| 16 | — | orf; Unknown function | |
| 16 | — | type III secretion apparatus protein | GOI |
| 16 | espP | putative exoprotein in pathogenicity island | GOI |
| 16 | hlyD | hemolysin transport | GOI |
| 17 | — | orf; Unknown function (Rhs Element | |
| | | Associated) | |
| 17 | stk | putative serine/threonine kinase encoded by | GOI |
| | | bacteriophage BP-933W | |
| 17 | — | orf; Unknown function | |
| 17 | — | putative protease encoded within prophage | |
| | | CP-933X | |
| 17 | — | orf; Unknown function | |
| 17 | ydbD | orf, hypothetical protein | |
| 17 | — | unknown protein encoded within prophage | |
| | | CP-933R | |
| 17 | ygeH | putative invasion protein | GOI |
| 17 | — | orf; Unknown function | |
| 17 | escT | type III secretion apparatus protein | |
| 18 | — | putative usher protein | |
| 18 | — | orf; Unknown function | |
| 18 | — | unknown | |
| 18 | — | orf; Unknown function | |
| 18 | yhiJ | orf, hypothetical protein | |
| 18 | yjbI | orf; Unknown function | |
| 18 | yjgL | orf; Unknown function | |
| 18 | — | putative invasin | GOI |
| 20 | — | orf; Unknown function | |
| 20 | — | orf; Unknown function | |
| 20 | — | orf; Unknown function | |
| 20 | ypjA | putative ATP-binding component of a | |
| | | transport system | |
| 21 | — | orf; Unknown function | |
| 21 | — | orf; Unknown function (Rhs Element | |
| | | Associated) | |
| 22 | — | unknown protein encoded by prophage | |
| | | CP-933X | |
| 22 | — | orf; Unknown function | |
| 22 | — | orf; Unknown function | |
| 23 | — | putative enterotoxin | GOI |
| 23 | — | orf; Unknown function | |
| 24 | — | orf; Unknown function | |
| 25 | — | putative invasin | GOI |
| 25 | wzy | O antigen polymerase | |
| 28 | — | orf; Unknown function | |
| 30 | hlyB | hemolysin transport | GOI |
| 31 | — | orf; Unknown function | |
| 31 | — | orf; Unknown function | |
| 31 | hlyA | hemolysin toxin pro | GOI |
| 36 | wzx | O antigen flippase Wzx | |
| 45 | — | unknown protein encoded by prophage | |
| | | CP-933N | |
| 159 | L7095 | putative cytotoxin | GOI |
| 51 Genes | | | |

27.5% GOI
*99 percentile ATA codon number used as statistical threshold.

TABLE 4c
EFL of ATG codon in E. coli O157.

| Frequency | | | |
|---|---|---|---|
| (#ATG/1000)* | Gene | Description | GOI? |
| 74.47 | — | unknown protein encoded within prophage CP-933V | |
| 75 | — | partial repeat of corA | |
| 75.47 | — | orf; Unknown function | |
| 75.76 | yjeT | orf, hypothetical protein | |
| 75.95 | — | orf; Unknown function | |
| 76.19 | fliE | flagellar biosynthesis; basal-body component, possibly at (MS- | GOI |
| | | ring)-rod junction | |
| 76.92 | celC | PEP-dependent phosphotransferase enzyme III for cellobiose, | |
| | | arbutin and salicin | |
| 76.92 | L7051 | hypothetical protein | |
| 76.92 | L7061 | hypothetical protein | |
| 77.46 | yecN | orf, hypothetical protein | |
| 77.78 | fliQ | flagellar biosynthesis | GOI |
| 77.92 | — | orf, hypothetical protein | |
| 79.55 | araH_A | partial high-affinity L-arabinose transport system; membrane | |
| | | protein, fragment 1 | |
| 80 | yecF | orf, hypothetical protein | |
| 80 | — | unknown protein encoded within prophage CP-933V | |
| 80 | — | orf; Unknown function | |
| 80.65 | — | putative Q antiterminator of prophage CP-933N | |
| 80.68 | eutH | ethanolamine utilization; homologue of Salmonella putative | |
| | | transport protein | |
| 80.88 | yidQ | orf; Unknown function | |
| 81.63 | — | orf; Unknown function | |
| 81.82 | ybaB | orf, hypothetical protein | |
| 81.82 | — | orf; Unknown function | |
| 83.33 | — | orf; Unknown function | |
| 84.51 | — | orf, hypothetical protein | |
| 85.11 | — | orf; Unknown function | |
| 85.11 | — | orf; Unknown function | |
| 85.11 | yceO | orf; Unknown function | |
| 85.71 | — | unknown protein encoded by prophage CP-933N | |
| 85.71 | — | unknown protein encoded within prophage CP-933U | |
| 85.71 | — | orf; Unknown function | |
| 86.42 | — | orf, hypothetical protein | |
| 88.24 | — | orf; Unknown function | |
| 90.91 | — | orf; Unknown function | |
| 98.04 | — | unknown protein encoded by prophage CP-933O | |
| 98.04 | — | orf; Unknown function | |
| 100 | — | orf; Unknown function | |
| 100 | cyoD | cytochrome o ubiquinol oxidase subunit IV | |
| 100 | — | orf; Unknown function (Rhs Element Associated) | |
| 100 | atpE | membrane-bound ATP synthase, F0 sector, subunit c | |
| 111.11 | — | orf; Unknown function | |
| 111.11 | — | orf; Unknown function | |
| 111.11 | — | orf Unknown function | |
| 116.28 | — | orf; Unknown function | |
| 116.28 | — | orf; Unknown function | |
| 117.65 | — | unknown protein encoded by prophage CP-933K | |
| 117.65 | — | orf, hypothetical protein | |
| 121.21 | — | orf; Unknown function | |
| 142.86 | — | orf; Unknown function | |
| 142.86 | — | Amino terminal fragment of WrbA | |
| 142.86 | — | orf; Unknown function | |
| 153.85 | — | unknown protein encoded in prophage CP-933I | |
| 157.89 | — | orf; Unknown function | |
| 157.89 | — | orf; Unknown function | |
| 200 | — | orf; Unknown function | |
| 53 Genes | | | 2GOI |

3.7% GOI
*99 percentile ATG codon frequency used as statistical threshold.

TABLE 4d
EFL of CTG codon in E. coli O157.

| Frequency | | | |
|---|---|---|---|
| (#CTG/1000)* | Gene | Description | GOI? |
| 113.92 | ycdG | putative transport protein | |
| 114.01 | agaI_2 | putative galactosamine-6-phosphate isomerase | |
| 114.04 | L7060 | hypothetical protein | |
| 114.29 | yhbO | orf, hypothetical protein | |
| 114.29 | — | putative resistance protein | |
| 114.29 | napH | ferredoxin-type protein: electron transfer | |
| 114.83 | — | putative capsid assembly protein of prophage CP-933O | |
| 115.99 | — | putative oxidoreductase | |
| 118.01 | — | putative exonuclease encoded by prophage CP-933K | |
| 118.23 | — | putative helicase | |
| 118.71 | creD | tolerance to colicin E2 | |
| 120 | zwf | glucose-6-phosphate dehydrogenase | |
| 120 | nuoG | NADH dehydrogenase I chain G | |
| 120 | ygbD | putative oxidoreductase | |
| 120 | flgD | flagellar biosynthesis, initiation of hook assembly | |
| 120.48 | udk | uridine/cytidine kinase | |
| 120.48 | traX | acetylase for F pil | |
| 121.53 | yhcH | orf, hypothetical protein | |
| 122.07 | sfmF | putative fimbrial protein | |
| 123.29 | yheN | orf, hypothetical protein | |
| 124.42 | — | putative tail component of prophage CP-933R | |
| 125 | fliK | flagellar hook-length control protein | |
| 125 | — | putative oxidoreductase | |
| 125 | mltA | membrane-bound lytic murein transglycosylase A | |
| 126.58 | — | orf; Unknown function | |
| 126.98 | — | unknown protein encoded within prophage CP-933L | |
| 127.27 | mglC | methyl-galactoside transport and galactose taxis | |
| 128.68 | — | putative tail component of prophage CP-933X | |
| 129.31 | galT | galactose-1-phosphate uridylyltransferase | |
| 129.31 | — | orf; Unknown function | |
| 129.31 | srlE_1 | PTS system, glucitol/sorbitol-specific IIB component and second | |
| | | of two IIC components; frag | |
| 129.31 | — | orf; Unknown function | |
| 129.31 | ibpB | heat shock protein | |
| 129.31 | yibQ | orf; Unknown function | |
| 129.31 | yciV | putative enzymes | |
| 129.31 | espA | secreted protein EspA | GOI |
| 132.28 | espB | secreted protein EspB | GOI |
| 133.33 | — | orf; Unknown function | |
| 137.5 | ybdE | putative inner membrane component for iron transport | |
| 137.57 | — | putative type-1 fimbrial protein | GOI |
| 139.01 | ydjY | orf, hypothetical protein | |
| 144.58 | pabC | 4-amino-4-deoxychorismate lyase | |
| 144.93 | ompC | outer membrane protein 1b (Ib;c) | |
| 144.93 | — | unknown protein encoded within prophage CP-933L | |
| 144.93 | trxA | thioredoxin 1 | |
| 144.93 | ytfQ | putative LACI-type transcriptional regulator | |
| 156.25 | terF_2 | partial putative phage inhibition, colicin resistance and tellurite | |
| | | resistance protein | |
| 160.71 | yojI | putative ATP-binding component of a transport system | |
| 160.71 | atpE | membrane-bound ATP synthase, F0 sector, subunit c | |
| 160.71 | nrdG | anaerobic ribonucleotide reductase activating protein | |
| 160.71 | prkB | probable phosphoribulokinase | |
| 225.81 | — | orf; Unknown function | |
| 425 | — | orf; Unknown function | |
| 54 Genes | | | 3GOI |

5.6% GOI
*99 percentile CTG codon frequency used as statistical threshold.

### Example 2.

Identification of possible genes of interest (PGOIs) by the methods described above. The EFL for ATA codons for E. coli O157 ORFs were generated as described herein. 99^{th} percentile results are shown in this example. See, Table 4e. PGOIs were also identified as described. Specifically, unknown ORFs identified as PGOIs (GenBank Protein ID numbers 12513990 and 12514510, i.e., conceptual translation products or “virtual proteins” of DNA ORFs) exhibit the highest ATA codon frequency of all ORFs in E. coli O157. Further analysis reveals the presence of leucine/isoleucine zipper motifs which are rare in eubacterial proteins (involving the ileX anticodon) but common in eukaryotic proteins involved in transcriptional regulation.

TABLE 4e
Identification of PGOIs EFL of ATA codon in E. coli O157.

| Frequency | | | | |
|---|---|---|---|---|
| (# ATA/ | | | | |
| 1000)* | PID | Gene | Description | PGOI? |
| 38.79 | 12516720 | — | orf; Unknown function | |
| 38.96 | 12514298 | — | unknown protein encoded by | |
| | | | bacteriophage BP-933W | |
| 39.06 | 12518451 | — | orf; Unknown function | |
| 39.22 | 12513956 | — | orf; Unknown function | |
| 39.22 | 12514473 | — | orf; Unknown function | |
| 39.47 | 12514196 | — | unknown protein encoded by | |
| | | | cryptic prophage CP-933M | |
| 39.47 | 12516387 | — | unknown protein encoded | |
| | | | within prophage CP-933V | |
| 39.47 | 12512948 | — | orf; Unknown function | |
| 39.68 | 12515296 | — | orf; Unknown function | |
| 39.68 | 12516291 | — | orf, hypothetical protein | |
| 40 | 12518552 | tnaL | tryptophanase leader peptide | |
| 40 | 12513062 | — | orf; Unknown function | |
| 40 | 12516085 | — | orf; Unknown function | |
| 40.16 | 12516226 | wbdO | glycosyl transferase | |
| 40.18 | 12518565 | — | orf; Unknown function | |
| 40.27 | 12517351 | — | putative 2-component | |
| | | | transcriptional regulator | |
| 40.4 | 12512947 | — | orf; Unknown function | |
| 40.48 | 12515215 | — | orf; Unknown function | |
| 40.54 | 12518549 | — | orf; hypothetical protein | |
| 40.75 | 12513054 | — | unknown protein encoded in | |
| | | | prophage CP-933I | |
| 40.82 | 12515240 | — | unknown protein associated | |
| | | | with Rhs element | |
| 40.82 | 12517482 | yqgB | orf, hypothetical protein | |
| 41.1 | 12517349 | — | orf, hypothetical protein | |
| 41.28 | 12516088 | — | unknown protein encoded | |
| | | | within prophage CP-933U | |
| 41.82 | 12517531 | — | putative enterotoxin | |
| 42.25 | 12515299 | — | orf; Unknown function | |
| 42.3 | 12515381 | — | unknown protein encoded | |
| | | | within prophage CP-933R | |
| 42.43 | | hlyB | hemolysin transport | |
| 42.55 | 12515363 | — | unknown protein encoded | |
| | | | within prophage CP-933R | |
| 42.68 | 12514395 | — | orf; Unknown function | |
| 43.01 | 12514677 | xisN | putative excisionase for | |
| | | | prophage | |
| 43.17 | 12513111 | — | orf; Unknown function | |
| 43.22 | 12514796 | — | unknown protein encoded by | |
| | | | prophage CP-933X | |
| 43.48 | 12515201 | rpsV | 30S ribosomal subunit | |
| | | | protein S22 | |
| 43.48 | 12516198 | — | orf; hypothetical protein | |
| 43.48 | 12518173 | — | orf; Unknown function | |
| 43.9 | 12518478 | — | orf; Unknown function | |
| 44.3 | 12512705 | — | orf; Unknown function | |
| 44.44 | 12517352 | — | orf; Unknown function | |
| 44.44 | 12513103 | — | orf, hypothetical protein | |
| 44.44 | 12517537 | — | orf; Unknown function | |
| 44.55 | 12515996 | — | putative serine acetlyl- | |
| | | | transferase of prophage | |
| | | | CP-933T | |
| 44.64 | 12517371 | — | type III secretion apparatus | |
| | | | protein | |
| 45 | 12518479 | — | orf; Unknown function | |
| 45.45 | 12514758 | — | unknown protein encoded by | |
| | | | prophage CP-933C | |
| 45.98 | 12514760 | — | unknown protein encoded by | |
| | | | prophage CP-933C | |
| 45.98 | 12515405 | ydaC | unknown protein encoded | |
| | | | within prophage CP-933R | |
| 45.98 | 12516228 | wbdN | glycosyl transferase | |
| 46.05 | 12513889 | — | orf; Unknown function | |
| 46.08 | 12517051 | — | orf; Other or unknown | |
| | | | (Phage or Prophage Related) | |
| 46.15 | | L7051 | hypothetical protein | |
| 46.3 | 12518480 | — | orf; Unknown function | |
| 46.3 | 12515023 | — | unknown protein encoded by | |
| | | | prophage CP-933O | |
| 46.36 | 12517365 | — | type III secretion apparatus | |
| | | | protein | |
| 46.51 | 12518691 | — | orf; Unknown function | |
| 46.51 | | hlyC | hemolysin transport | |
| 46.88 | 12514316 | stx2A | shiga-like toxin II A subunit | |
| | | | encoded by bacteriophage | |
| | | | BP-933W | |
| 47.17 | 12516336 | yehK | orf; Unknown function | |
| 47.62 | 12515011 | — | unknown protein encoded by | |
| | | | prophage CP-933O | |
| 47.62 | 12517350 | — | orf; Unknown function | |
| 47.62 | 12515746 | — | orf; Unknown function | |
| 47.97 | 12514743 | — | unknown protein encoded by | |
| | | | prophage CP-933N | |
| 48.71 | 12514297 | stk | putative serine/threonine | |
| | | | kinase encoded by | |
| | | | bacteriophage BP-933W | |
| 49.18 | 12519199 | — | orf; Unknown function | |
| 50 | 12513997 | — | orf; Unknown function | |
| 50 | 12514409 | — | orf; Unknown function | |
| 50 | 12514517 | — | orf; Unknown function | |
| 50.16 | | L7095 | putative cytotoxin | |
| 50.46 | 12518477 | escR | type III secretion apparatus | |
| | | | protein | |
| 51.16 | 12513917 | — | orf; Unknown function | |
| 51.16 | 12514434 | — | orf; Unknown function | |
| 51.28 | 12518780 | — | orf; Unknown function | |
| 51.66 | 12514396 | — | putative regulator; Not | PGOI |
| | | | classified | |
| 51.72 | 12519105 | — | orf; Unknown function | |
| 52.63 | 12513923 | — | orf; Unknown function | |
| 52.63 | 12514440 | — | orf; Unknown function | |
| 52.63 | 12515362 | — | unknown protein encoded | |
| | | | within prophage CP-933R | |
| 53.19 | 12513940 | — | orf; Unknown function | |
| 53.19 | 12514460 | — | orf; Unknown function | |
| 53.57 | 12513709 | — | unknown protein encoded by | |
| | | | prophage CP-933K | |
| 54.05 | 12517060 | — | unknown protein encoded by | |
| | | | prophage CP-933Y | |
| 54.79 | 12513989 | — | orf; Unknown function | |
| 54.79 | 12514506 | — | orf; Unknown function | |
| 54.88 | 12517343 | — | orf; Unknown function | |
| 56.41 | 12514742 | — | unknown protein encoded by | |
| | | | prophage CP-933N | |
| 57.47 | 12517354 | — | orf; Unknown function | |
| 58.56 | 12517367 | — | putative integral membrane | |
| | | | protein-component of type III | |
| | | | secretion apparatus | |
| 63.06 | 12516214 | wbdR | acetyl transferase; O-antigen | |
| | | | biosynthesis | |
| 63.29 | 12518846 | — | orf; Unknown function | |
| 63.29 | 12516227 | wzy | O antigen polymerase | |
| 65.64 | 12518475 | escT | type III secretion apparatus | |
| | | | protein | |
| 66.67 | 12513611 | — | orf; Unknown function (Rhs | |
| | | | Element Associated) | |
| 66.67 | 12517380 | — | orf; Unknown function | |
| 67.57 | 12513607 | — | rhsC protein in rhs element | |
| 75 | 12516312 | — | orf; Unknown function | |
| 75.27 | 12513404 | ybbV | orf, hypothetical protein | |
| 75.47 | 12514732 | — | unknown protein encoded by | |
| | | | prophage CP-933N | |
| 76.92 | 12513724 | — | unknown protein encoded by | |
| | | | prophage CP-933K | |
| 77.59 | 12516225 | wzx | O antigen flippase Wzx | |
| 78.43 | 12518473 | — | orf; Unknown function | |
| 80.46 | 12513391 | ybbD | orf, hypothetical protein | |
| 81.3 | 12513957 | — | orf; Unknown function | |
| 81.3 | 12514474 | — | orf; Unknown function | |
| 85.71 | 12518551 | — | orf; Unknown function | |
| 88.89 | 12518476 | escS | type III secretion apparatus | |
| | | | protein | |
| 91.74 | 12513608 | ybfB | orf, hypothetical protein | |
| 95.89 | 12518481 | — | orf; Unknown function | |
| 212.12 | 12513990 | — | orf; Unknown function | PGOI |
| 212.12 | 12514510 | — | orf; Unknown function | PGOI |
| 109 Genes | | | | |

*98 percentile ATA codon frequency used as statistical threshold.

Identification of Specific Codons Greatly Enriched in Pathogenicity Elements

While large CDI and RCDI scores indicate that, on average, the codons of certain genes may be divergent from those of the host organisms, such indices do not indicate which codons are aberrant. A script was written to identify the codons in each gene whose frequencies diverge the greatest from those of the host genome. Results for several virulence genes are shown in Table 5.

TABLE 5
Identification of Codons with Increased Codon Frequencies in
Virulence Genes.

| | | Frequency | | | |
|---|---|---|---|---|---|
| | | in | Frequency in | | |
| | Codon | Gene | Genome | Difference | % increase |
| C. jejuni | | | | | |
| pVir | | | | | |
| comB2 | GAC | 16.81 | 4.18 | 12.63 | 302.15% |
| | ACG | 8.4 | 2.51 | 5.89 | 234.66% |
| | CAG | 8.4 | 2.89 | 5.51 | 190.66% |
| | CAC | 8.4 | 3.16 | 5.24 | 165.82% |
| | CAA | 64.43 | 28.31 | 36.12 | 127.59% |
| | ACC | 14.01 | 6.17 | 7.84 | 127.07% |
| | TAC | 11.2 | 5.01 | 6.19 | 123.55% |
| | CCC | 2.8 | 1.31 | 1.49 | 113.74% |
| comB3 | AAC | 29.02 | 8.87 | 20.15 | 227.17% |
| | ACG | 7.92 | 2.51 | 5.41 | 215.54% |
| | TAC | 13.19 | 5.01 | 8.18 | 163.27% |
| | CTG | 2.64 | 1.08 | 1.56 | 144.44% |
| | CAA | 63.32 | 28.31 | 35.01 | 123.67% |
| | TCA | 21.11 | 9.99 | 11.12 | 111.31% |
| virB11 | AGG | 9.43 | 2.73 | 6.7 | 245.42% |
| | AAC | 28.3 | 8.87 | 19.43 | 219.05% |
| | CAC | 9.43 | 3.16 | 6.27 | 198.42% |
| | CTG | 3.14 | 1.08 | 2.06 | 190.74% |
| | ACG | 6.29 | 2.51 | 3.78 | 150.60% |
| | CCC | 3.14 | 1.31 | 1.83 | 139.69% |
| | CTC | 6.29 | 2.71 | 3.58 | 132.10% |
| | TGT | 18.87 | 8.63 | 10.24 | 118.66% |
| | CAG | 6.29 | 2.89 | 3.4 | 117.65% |
| | AGA | 34.59 | 16.02 | 18.57 | 115.92% |
| | TCG | 3.14 | 1.55 | 1.59 | 102.58% |
| P. aer | | | | | |
| CTX | | | | | |
| ctx | TCT | 10.49 | 0.83 | 9.66 | 1163.86% |
| | TTA | 3.5 | 0.28 | 3.22 | 1150.00% |
| | ACT | 17.48 | 1.66 | 15.82 | 953.01% |
| | ACA | 6.99 | 0.81 | 6.18 | 762.96% |
| | AGA | 3.5 | 0.48 | 3.02 | 629.17% |
| | ATT | 17.48 | 2.86 | 14.62 | 511.19% |
| | TTT | 10.49 | 1.72 | 8.77 | 509.88% |
| | TCA | 3.5 | 0.58 | 2.92 | 503.45% |
| | AGT | 13.99 | 2.62 | 11.37 | 433.97% |
| | GTT | 13.99 | 2.72 | 11.27 | 414.34% |
| | CAA | 31.47 | 6.25 | 25.22 | 403.52% |
| | CTA | 6.99 | 1.4 | 5.59 | 399.29% |
| | AAT | 17.48 | 3.73 | 13.75 | 368.63% |
| | TAT | 24.48 | 5.26 | 19.22 | 365.40% |
| | GGA | 17.48 | 4.16 | 13.32 | 320.19% |
| | TTG | 34.97 | 8.74 | 26.23 | 300.11% |
| | ATA | 3.5 | 0.95 | 2.55 | 268.42% |
| | CTT | 10.49 | 3.09 | 7.4 | 239.48% |
| | AAC | 48.95 | 22.59 | 26.36 | 116.69% |
| | GCA | 10.49 | 4.85 | 5.64 | 116.29% |
| | TGG | 31.47 | 14.82 | 16.65 | 112.35% |
| HPY199 | | | | | |
| cagT | AGT | 39.15 | 9.66 | 29.49 | 305.28% |
| | CAG | 17.79 | 5.52 | 12.27 | 222.28% |
| | GCA | 21.35 | 6.8 | 14.55 | 213.97% |
| | CCA | 14.23 | 4.85 | 9.38 | 193.40% |
| | CTG | 10.68 | 4.28 | 6.4 | 149.53% |
| | AAG | 489.82 | 20.68 | 29.14 | 140.91% |
| | TAC | 24.91 | 11.43 | 13.48 | 117.94% |
| | AGA | 17.79 | 8.75 | 9.04 | 103.31% |
| E. coli | | | | | |
| 933W | | | | | |
| Bacterio- | | | | | |
| phage | | | | | |
| stk | AGA | 25.86 | 2.13 | 23.73 | 1114.08% |
| | ATA | 48.85 | 4.39 | 44.46 | 1012.76% |
| | AGG | 11.49 | 1.25 | 10.24 | 819.20% |
| | CTA | 22.99 | 3.91 | 19.08 | 487.98% |
| | TAT | 48.85 | 16.21 | 32.64 | 201.36% |
| | CAT | 37.36 | 12.96 | 24.4 | 188.27% |
| | ACA | 20.11 | 7.12 | 12.99 | 182.44% |
| | TGT | 14.37 | 5.19 | 9.18 | 176.88% |
| | TTA | 34.48 | 13.92 | 20.56 | 147.70% |
| | AAT | 43.1 | 17.78 | 25.32 | 142.41% |
| | TCA | 17.24 | 7.2 | 10.04 | 139.44% |
| | CCA | 20.11 | 8.45 | 11.66 | 137.99% |
| | TCT | 20.11 | 8.47 | 11.64 | 137.43% |
| | GGA | 17.24 | 8.01 | 9.23 | 115.23% |
| stxA2 | ATA | 47.02 | 4.39 | 42.63 | 971.07% |
| | AGA | 18.81 | 2.13 | 16.68 | 783.10% |
| | AGG | 6.27 | 1.25 | 5.02 | 401.60% |
| | ACA | 34.48 | 7.12 | 27.36 | 384.27% |
| | TCA | 21.94 | 7.2 | 14.74 | 204.72% |
| | AGT | 25.08 | 8.81 | 16.27 | 184.68% |
| | ACT | 21.94 | 8.98 | 12.96 | 144.32% |
| | AAT | 40.75 | 17.78 | 22.97 | 129.19% |
| | TCT | 18.81 | 8.47 | 10.34 | 122.08% |
| | TCC | 18.81 | 8.65 | 10.16 | 117.46% |
| S. flex | | | | | |
| virF | ATA | 64.64 | 4.39 | 60.25 | 1372.44% |
| | AGG | 15.21 | 1.25 | 13.96 | 1116.80% |
| | AGA | 22.81 | 2.13 | 20.68 | 970.89% |
| | TCT | 41.83 | 8.47 | 33.36 | 393.86% |
| | TCA | 30.42 | 7.2 | 23.22 | 322.50% |
| | TTA | 45.63 | 13.92 | 31.71 | 227.80% |
| | CTT | 30.42 | 11.04 | 19.38 | 175.54% |
| | TAT | 41.83 | 16.21 | 25.62 | 158.05% |
| | GAG | 41.83 | 17.87 | 23.96 | 134.08% |
| | AAG | 22.81 | 10.35 | 12.46 | 120.39% |
| | CGA | 7.6 | 3.57 | 4.03 | 112.89% |
| | AAA | 68.44 | 33.67 | 34.77 | 103.27% |
| S. typhi | | | | | |
| spaL | AGG | 17.89 | 1.25 | 16.64 | 1331.20% |
| | CGA | 38.77 | 3.57 | 35.2 | 985.99% |
| | AGA | 20.87 | 2.13 | 18.74 | 879.81% |
| | GGA | 35.79 | 8.01 | 27.78 | 346.82% |
| | CGG | 23.86 | 5.44 | 18.42 | 338.60% |
| | TGC | 26.84 | 6.5 | 20.34 | 312.92% |
| | TGT | 14.91 | 5.19 | 9.72 | 187.28% |
| | CCC | 14.91 | 5.51 | 9.4 | 170.60% |
| | TCG | 20.87 | 8.96 | 11.91 | 132.92% |
| E. coli | | | | | |
| pO157 | | | | | |
| hlyD | AGA | 18.75 | 2.13 | 16.62 | 780.28% |
| | ATA | 33.33 | 4.39 | 28.94 | 659.23% |
| | AGG | 6.25 | 1.25 | 5 | 400.00% |
| | ACA | 31.25 | 7.12 | 24.13 | 338.90% |
| | TCT | 31.25 | 8.47 | 22.78 | 268.95% |
| | CTT | 35.42 | 11.04 | 24.38 | 220.83% |
| | CCT | 18.75 | 7.03 | 11.72 | 166.71% |
| | GTT | 47.92 | 18.31 | 29.61 | 161.71% |
| | GTA | 27.08 | 10.92 | 16.16 | 147.99% |
| | TCA | 16.67 | 7.2 | 9.47 | 131.53% |
| putative | ATA | 50.16 | 4.39 | 45.77 | 1042.60% |
| cytotoxin | | | | | |
| | AGA | 19.24 | 2.13 | 17.11 | 803.29% |
| | TCA | 29.65 | 7.2 | 22.45 | 311.81% |
| | ACA | 28.08 | 7.12 | 20.96 | 294.38% |
| | AGG | 4.73 | 1.25 | 3.48 | 278.40% |
| | AAT | 65.93 | 17.78 | 48.15 | 270.81% |
| | TTA | 42.9 | 13.92 | 28.98 | 208.19% |
| | CTA | 10.73 | 3.91 | 6.82 | 174.42% |
| | AGT | 23.34 | 8.81 | 14.53 | 164.93% |
| | TCT | 22.08 | 8.47 | 13.61 | 160.68% |
| | TAT | 37.85 | 16.21 | 21.64 | 133.50% |
| | GGA | 17.03 | 8.01 | 9.02 | 112.61% |
| STMD1 | | | | | |
| Control | | | | | |
| Region | | | | | |
| fadB | GGC | 56.24 | 29.69 | 26.55 | 89.42% |
| | GTC | 27.43 | 15.29 | 12.14 | 79.40% |
| | CCG | 41.15 | 23.24 | 17.91 | 77.07% |
| aarF | CGA | 10.99 | 3.57 | 7.42 | 207.84% |
| | CGG | 12.82 | 5.44 | 7.38 | 135.66% |
| | CCT | 16.48 | 7.03 | 9.45 | 134.42% |
| | TTG | 25.64 | 13.69 | 11.95 | 87.29% |
| ilvA | GCG | 81.71 | 33.71 | 48 | 142.39% |
| | TGC | 13.62 | 6.5 | 7.12 | 109.54% |
| | GGC | 54.47 | 29.69 | 24.78 | 83.46% |

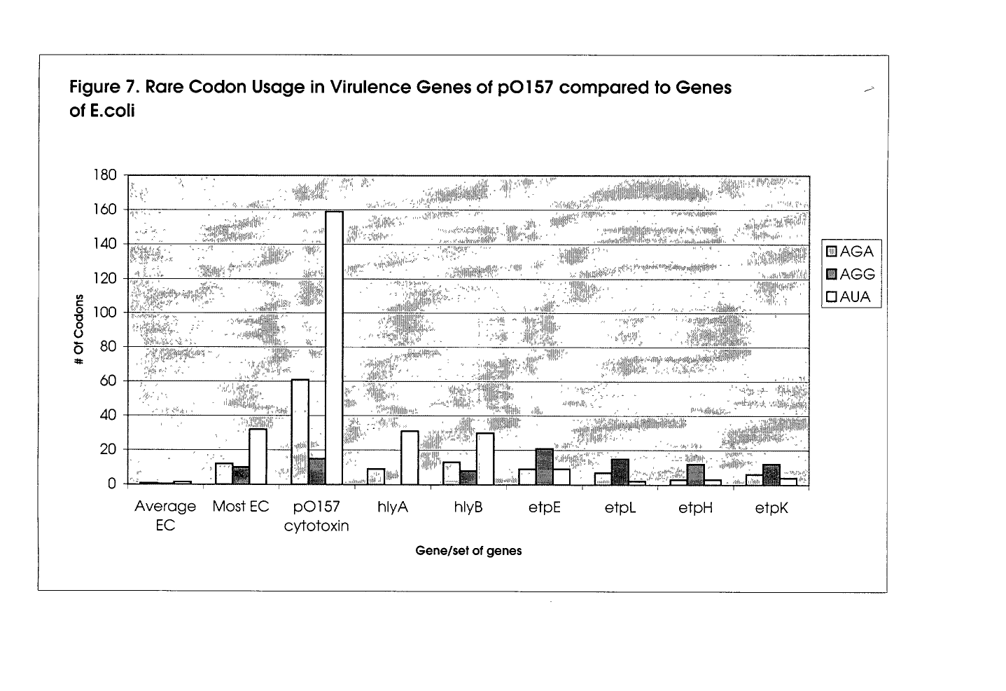

As can be seen in Table 5, the frequencies of codon usage of three rare E. coli codons, AUA, AGG, and AGA were greatly increased in several pathogenic E. coli and Shigella virulence genes. Whereas there are only 6 genes in E. coli greater than 250 amino acids that have an AUA codon frequency greater than 45/1000 codons, the stk and stxA2 genes of the 933W bacteriophage, virF of Shigella and the putative cytotoxin of pO157 have frequencies which exceed this mark. The large putative cytotoxin of pO157 actually has 159 AUA codons and 61 AGA codons, 5 times more of each codon than is found in any other E. coli protein while hlyA and hlyB, also found on pO157, have more AUA codons than any other E. coli protein except one (see, FIG. 7). Similarly, 4 members of the type II secretion apparatus found on pO157, along with the putative cytotoxin and two hypothetical genes, all have more AGG codons than any other gene in E. coli K12. Although many codons were present in virulence genes at frequencies greater than 10 times that of the average in the host genomes, no such enrichment in codon frequencies was observed for genes in the STMD1 and STMF1 control regions of Salmonella typhi. See, Table 5. Of course, in organisms wherein the specific virulence genes are not yet determined, the increased presence of such rare usage codons in a gene can flag it for examination to determine if it is relevant to virulence (see, below for examples of such methods).

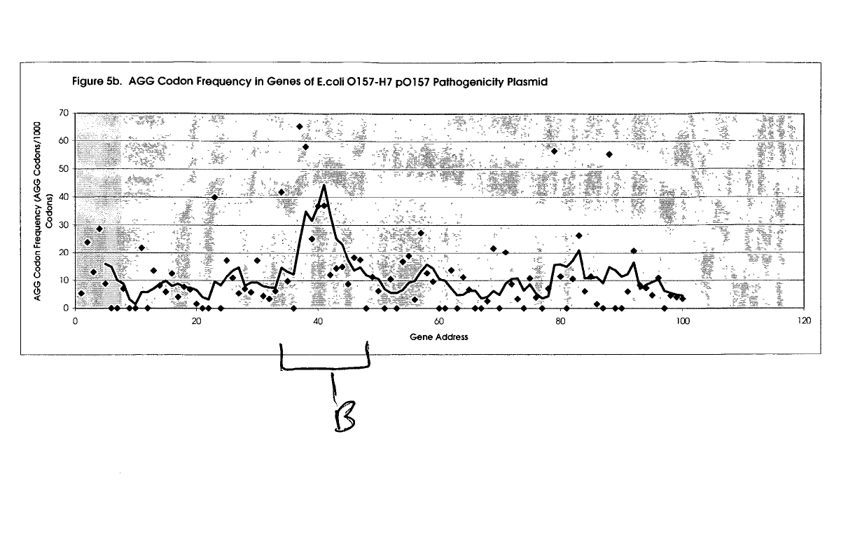

AUA and AGG codon frequency are graphed according to ORF position in FIG. 5 for pO157 and a moving average line with a period of 5 is drawn. Frequencies for both codons appear enriched for certain regions of the pO157 plasmid. These enriched regions appear at peak A for AUA and peak B for AGG. These peaks correspond to the hemolysin toxin and transporters (peak A) and type II secretion apparatus (peak B) of E. coli O157:H7. Similar enriched regions were found for AUA in the LEE pathogenicity island and the 933W bacteriophage, also of E. coli O157:H7, and correspond to the genes for the type II secretion pathway and the stk serine-threonine kinase region respectively (data not shown).

Interestingly, the 933W bacteriophage which also infects E. coli O157:H7 contains three tRNAs: one for the rare isoleucine codon AUA and one each for the rare arginine codons AGA and AGG, suggesting that these tRNAs may otherwise exist at levels that limit translation of these genes.

The rare isoleucine tRNA, ileX, has a CAU anticodon that is known to be modified to k2C in E. coli, B. subtilis, and Mycoplasma capricolum. See, e.g., Sprinzl, M., et al. (1998) Nuc Acids Res 26(1):148-53. This modification has been demonstrated to be essential for the proper translation of AUA as isoleucine in these organisms and is also thought to be an identity element for the isoleucine tRNA synthetase. See, e.g., Nureki, O., et al., (1994) J Mol Biol 236(3):710-24. If the lysidine modification, k2C, is not essential for cell viability, it will likely be essential for expression of stk, the typeIII secretion apparatus, the hemolysin toxin and transporter, the shiga-toxin 2A subunit, and other virulence factors in pathogenic E. coli and other bacteria due to an extremely high frequency of AUA codons in these genes. Thus, it is a possible target for modification through the methods herein (e.g., to attenuate virulence of such pathogenic organisms).

Similarly, the t6A modification is present in the E. coli ArgU tRNA at position 37 and may increase the efficiency of translation in a manner similar to i6A which occurs at the same position in the tRNA. Very high frequencies of the codons AGA and AGG, which are recognized by ArgU in E. coli, have been found relative to the rest of the E. coli genome in a variety of pathogenicity genes, including stk, stxA2, hlyD, and the large putative cytotoxin of E. coli O157:H7. Previous studies have shown that the ArgU tRNA may be present at levels that modulate the translation of the int gene from lambda phage, which also has a high frequency of AGA and AGG codons. See, e.g., Zahn, K., et al. (1996) Mol Microbiol 21(1):69-76. Due to their increased dependence on AGA and AGG codons, inhibition of the t6A enzyme may prevent proper translation of these genes. Furthermore, increased AGA and AGG frequencies are also found in virB 11 of C. jejuni pVir, the ctx cytotoxin gene of P. aeruginosa ΦCTX, cagT of the H. pylori cag pathogenicity island, virF of Shigella, and spaL of Salmonella, although it is not clear if the t6A modification or some other modification is present in the arginine tRNA of these organisms to help decode these codons. If t6A, or some other modification, is present in these tRNAs and improves the efficiency of translation, it is likely that expression of these genes would be greatly impaired in a t6A deficient cell. Again, these specific rare codon usages in virulence genes (and the necessary tRNA modifications needed to utilize them) are optional targets for attenuating virulence.

Since codon usage in pathogenicity elements is often significantly different from that of the host genome, factors that influence translation may effect the translation of these elements more dramatically than genes normally encoded by the host. The methods of the present invention provide a mechanism for determining these differences in codon usage, as well as identifying targets for compositions or drugs designed to take advantage of these differences. tRNA modifications that effect translation may have a greater impact on translation of virulence genes than on other genes. This has been shown for miaA and tgt mutants in E. coli, Shigella, and Agrobacterium and can be applied to alternative tRNA modifications, for example, the lysidine modification necessary for decoding AUA as isoleucine, and the cmo5U and mnm5U modifications which effect the wobble pairings of several tRNAs. Further characterization of modifications, such as t6A, a modification similar to the i6A modification performed by miaA, may also reveal a role in the translation of genes and may be a important factor in the translation of virulence factors.

Finally, tRNA modifying enzymes (or transfer RNA modification enzymes), TMEs, may prove to be an exciting class of drug targets for the methods of the present invention for several reasons. Several TME mutants, including trmA and yfhC, the E. coli tRNA (adenosine-34) deaminase have been demonstrated to be essential for cell viability (see, e.g., Persson, B., et al., (1992) Proc Natl Acad Sci USA 89(9):3995-8), although this effect appears to be independent of the tRNA modification function of the trmA enzymes. Two others, tgt and miaA, have been proven to be essential for the virulent phenotype of Shigella while miaA has also been demonstrated to be essential for virulence in pathogenic E. coli and contributes to virulence in Agrobacterium. Other enzymes, such as those responsible for the k2C and t6A modifications, may also prove to be essential for cell viability. If these enzymes prove to be dispensable for cell survival, they will likely be essential for translation of many virulence factors, as are tgt and miaA, due to the remarkable increase in the frequency of codons recognized by tRNAs which require these modifications for proper function. The possibility also exists that previously identified virulence-associated loci of unknown function may prove to encode TMEs as was the case for tgt and miaA in Shigella and Agrobacterium.

While previous studies have demonstrated a role for tRNAs and tRNA modification in virulence gene translation, the current invention utilizes tRNAs and tRNA modification in virulence gene translation as a controlling point in virulence factor expression due to the anomalous codon usage of many known virulence genes. The methods of the present invention optionally include further actions, such as sequencing of pathogenic bacteria, tRNA sequencing, and bioinformatics.

Effects of Reduced Levels of Functional TRNA

Recently it was discovered that the insertion and excision sites of PAI-1 and PAI-2 in UPEC (uropathogenic) strain 536 were leuX and seIC, respectively. See, e.g., Blum, G., et al., (1994) Infection and Immunity 62:606-614. Upon excision from the chromosome, the PAIs removed the 3′ ends of the tRNAs, leaving the cells without functional copies of leuX and selC tRNAs. While there is no other tRNA capable of decoding the UGA stop/selenocysteine codon translated by selC, leuZ is able to “wobble” to recognize the UUG codon recognized by leuX. However, while the leuX-cells are still viable, they are avirulent, serum sensitive and fail to produce a flagella, type 1 fimbria, or enterobactin (see, Table 3 and Ritter, A., et al., (1995) Mol Microbiol 17(1):109-21). In addition they are unable to survive in mouse bladder mucus (see, e.g., Dobrindt, U., et al., (1998) FEMS Microbiol Lett. 162(1):125-41) and fail to colonize the large intestine of mice when fed together with wild-type cells. Remarkably, all these phenotypes are due to the lack of leuX and not the loss of the PAI-1. Complementation with a plasmid encoding leuX, but not PAI-1 or PAI-2, restored the wild-type phenotype. In addition, a random screen to identify clones that resulted in recolonization of the mouse intestine resulted in the isolation of a 6.5 kb fragment containing leuX. Further characterization of this clone revealed that the leuX gene was the essential factor that restored the colonization phenotype. See, e.g., Newman, J., et al., (1994) FEMS Microbiol Lett 122(3):281-7. Furthermore, the loss of type 1 fimbria was found to be due to poor translation of the fimB protein. However, changing the five UUG leucine codons to CUG leucine codons resulted in full expression of fimB and the production of fimbria. See, Ritter, A., et al., (1997) Mol Microbiol 25(5):871-82. The reading of UUG codons by leuZ in leuX-UPEC due to wobble was sufficient to express the proteins required for survival and growth of the bacteria, but was insufficient to translate enough of the fimB protein, which has UUG codon frequencies close the average in E. coli, in order to make type 1 fimbria. The genes responsible for enterobactin and flagella production which are poorly translated in a leuX-strain have not yet been identified. However, it is interesting to note that entF has more UUG codons than all but 17 genes in E. coli 21 UUG codons), while several genes involved in flagellar biosynthesis, including fliP, fliQ, flhA, fhiA, and flhD have either many UUG codons or a high frequency of UUG codons. See, Table 6. The above illustrates the many possible targets/actions for attenuation of virulence of such organisms due to increased rare codon usage in virulence genes, etc.

TABLE 6
UUG Codon Frequency and Number in flagellar biosynthesis and
enterobactin genes possibly responsible for leuX knockout effect.

| | | Frequency | |
|---|---|---|---|
| | | per | # UUG |
| | | 1000 Codons | Codons |
| Average | | 13.82 | 4.34 |
| Frequency/Number | | | |
| in E. coli Genes | | | |
| Highest | | 83.87 | 36 |
| Frequency/Number | | | |
| in E. coli Genes | | | |
| 95 percentile of | | 39.43 | 12 |
| UUG usage | | | |
| flhA | flagellar biosynthesis; | 34.63 | 24 |
| | possible export of flagellar | | |
| | proteins | | |
| flhD | regulator of flagellar | 41.67 | 5 |
| | biosynthesis, acting on | | |
| | class 2 operons; trans- | | |
| | criptional initiation factor | | |
| fliP | flagellar biosynthesis | 52.85 | 13 |
| fliQ | flagellar biosynthesis | 66.67 | 6 |
| fhiA | flagellar biosynthesis | 20.69 | 12 |
| entF | ATP-dependent serine | 16.23 | 21 |
| | activating enzyme (may be | | |
| | part of enterobactin | | |
| | synthase as component F) | | |

In Shigella flexneri, the vacC virulence-associated chromosomal locus, identified by random Tn5 insertion mutagenesis, was found to encode the tgt tRNA modification enzyme, which catalyzes a step in queosine-34 (Q) biosynthesis. See, e.g., Durand, J., et al., (1994) J Bacteriol 176(15):4627-34. The Q modification appears to decrease the readthrough of UAA codons by Tyr tRNAs and may play other roles in maintaining faithful translation of other codons. Another TME, miaA, which catalyzes the production of the i6A modification at position 37, may increase translation efficiency by nearly 100-fold in some contexts and reduces strand slippage and stop codon readthrough. Durand et al. demonstrated that the reduced virulence of tgt mutants and avirulence of miaA mutants was due primarily to the poor expression of virF, a regulatory protein which controls transcription of multiple virulence factors including virG, mxiA, and the spa and ipa operons which are involved in intracellular spreading and invasion of epithelial cells. They further showed that miaA was also essential for virulence phenotypes in Shigella dysenteriae type 3 strain, Shigella sonnei 65 strain, and in EIEC O152 indicating that this effect is conserved in other virulent enterobacteria. See, Durand, J., et al., (1997) J Bacteriol 179(18):5777-82. In the plant pathogen Agrobacterium tumefaciens, a transposon mutagenesis screen for chromosomal genes that influence expression of the vir virulence factor also resulted in the identification of its miaA homologue as a virulence factor. See, Gray, J., et al., (1992) J Bacteriol 174(4): 1086-98. Thus, two random mutagenesis screens identified two TMEs, tgt in Shigella flexneri and miaA in Agrobacterium, as virulence factors required for full pathogenicity.

tgt mutants in Shigella show similar growth rates as wild-type cells while miaA mutants grow 30-40% slower. While these modification enzymes are not essential for the survival of the bacteria, they are essential for full virulence, again, illustrating the basic concepts herein. A lack of the tRNA modifications produced by these enzyme may cause a reduction in the functional pool of tRNA due to a decrease in translation efficiency or a decrease in the stability and therefore the levels of tRNA as has recently been suggested for other modifications. See, e.g., Yasukawa, T., et al., (2000) J Biol Chem 275(6):4251-7. These results indicate that certain genes are more susceptible to problems in translation caused by a reduced level of functional tRNAs due to deletion of the tRNA or lack of a tRNA modification. Thus, they may be even more sensitive to modifications as described herein to attenuate virulence, etc.

Irregular Codon Usage May Make VirF Expression More Susceptible to miaA and tgt Knockouts

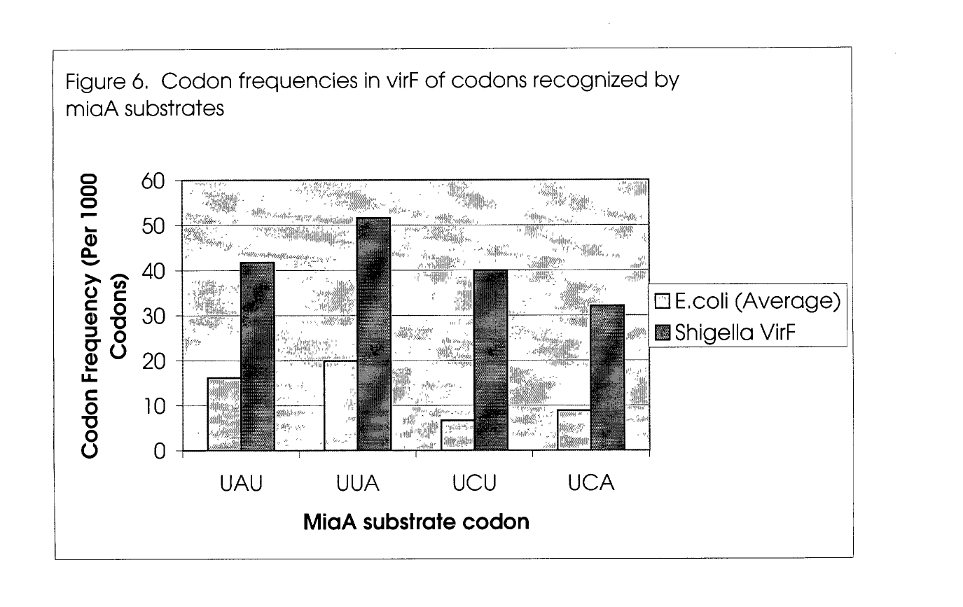

It seems straightforward that an increase in susceptibility to errors in translation due to reduced fidelity in decoding a particular codon would be greater in genes enriched in that particular codon. For example, the observed effect of miaA and tgt mutants on S. flexneri virF gene translation is optionally thus. Tgt and miaA modify different tRNAs with the exception of tRNATyr. Interestingly, the virF gene has a marked increase in the use of the UAU tyrosine codon as compared to the average in E. coli K12. E. coli K12 is used as the reference genome since evidence exists that E. coli and Shigella are actually different serovars of the same species. The average UAU codon frequency per 1000 codons in E. coli K12 is 16.17 whereas the frequency in virF is 41.79 (see, FIG. 6). In addition, the frequency of other codons decoded by miaA substrates is also dramatically increased. The frequency of the UUA leucine codon is increased from the E. coli average of 19.91 to 51.62 in virF while the serine codons UCU and UCA are increased from 6.64 and 8.85 to 40.0 and 32.07 codons per 1000 respectively. This dramatic increase in frequency of codons decoded by miaA tRNA substrates (UAU, UUA, UCU, UCA) and tgt tRNA substrates (UAU) is optionally the reason for virF's poor translation in tgt and miaA knockouts relative to other gene products.

Increased Frequency of Rare Codon AUA in E. coli Pathogen 0157

The frequency of the rare Ile codon AUA is elevated in the pathogen E. coli 0157. As explained herein, AUA enrichment is confined to 0157 genes that are not present in wild type E. coli. Concurrent expansion of the methionyl elongator tRNA genes in 0157 is, thus, most likely functionally related to the expression of its AUA rich gene set. In E. coli modification of elongator “methionyl” tRNA (em_tRNA) by conversion of anti-codon base C34 to lysidine is required for translation of the isoleucine codon AUA. The genome of E. coli 0157, an enteric pathogen, contains about 1500 genes not found in wild type E. coli (e.g., strain MG 1655). Many of these added genes are located in “pathogenicity islands” (see, above) and encode known virulence determinants.

When the codon distribution in the “virulence” gene set is compared to the shared gene set, it is seen that the rare isoleucyl codon AUA is dramatically over represented in the 0157 gene set. Genes in the 0157 set have an average AUA frequency per thousand codons (FTP) of 12.24. This is roughly twice the frequency of AUA in genes common to both E. coli MG1655 and E. coli 0157 (AUA FTP of 5.23 in MG1655 and 5.18 in 0157). A lysine modification of em_tRNA is required for translation of AUA. These genes have increased from 2 copies in the wild type genome to 10 copies in the pathogenic species. The elongator tRNA sequences are not identical, perhaps indicating acquisition by horizontal transfer. Nevertheless, known determinants for recognition of the em_tRNA substrate by Ile tRNA synthetase can be used to identify tRNAs likely to be lysinylated and to mediate translation of the Ile AUA codon. Of the 10 em_tRNAs, 8 match the Ile RS profile perfectly. Thus, expression of the 0157 virulence genes requires the translation of unusually large numbers of AUA codons and is therefore dependent on lysinylation of the expanded elongator methionyl tRNA set, inferring that lysinylation potentiates, and thus may regulate, virulence in this pathogen. Thus, as explained herein, a potential target for action against the pathogenic strain is optionally through enzymes, etc. required for this lysinylation. See, above.

Lysidine Modification of Anti-codon Position 34 in Specific Bacteria

Lysidine, or a similar modification of tRNA_{cau} at anti-codon position 34, is highly conserved in archaea and eubacteria, is essential to such organisms, and is probably mediated by an enzymatic activity. In several bacteria, isoleucyl tRNA_{cau} is absent. Instead, the cognate isoleucine codon AUA is translated by a “methionyl” tRNA, post-transcriptionally modified to lysidine at C at anti-codon position 34. This confers complete functional metamorphosis on the tRNA which, unmodified, reads the methionine codon AUG and is appropriately charged. To date, no gene or enzyme has been linked to lysinylation.

Comparative analysis of the tRNA distribution in 35 sequenced bacterial genomes reveals that the isoleucine tRNA_{cau} is never found. Moreover, methionyl tRNA are always present in sets of three or more copies in each species. This multiplication is unique among bacterial tRNA genes. Setting aside the initiator tRNAₘₑₜ, each set in every case contains at least two distinct tRNAₘₑₜ “siblings.” No detectable sequence motif exists to steer one sibling into the lysinylation pathway, yet, an enzymatic process ought to act on specific substrates. Pairwise disjunction analysis of the tRNAₘₑₜ sets reveals a site, position 44, which is consistently a different base, and which, therefore, distinguishes the siblings of each species. These sites of “conserved difference” are likely to be structural discriminators, possibly enzyme recognition sites on the tRNA. As such, although non-enzymatic or even autocatalytic modification remains a formal possibility, the discriminator sites provide the first evidence for a hypothetical lysinylation enzyme. To examine the essentiality of the modification, the tRNAₘₑₜ substrate of lysinylation in E. coli, ileX was knocked out, as was the ileX homologue in B. subtilis. Both knockouts proved lethal.

Thus, based upon the comparative distribution of tRNA genes, lysinylation, or some similar modification, is an apparent universal feature of bacterial life. The modification is essential. A spatially conserved discriminator site at position 44 distinguishes the elongator methionyl tRNA siblings in all bacteria and is an optional recognition site for a putative lysinylation enzyme.

Screening/Characterization of Identified Areas of Rare Codon Usage For Involvement in Pathogenesis/Virulence

As outlined above, in typical embodiments of the current invention, genes comprising areas of, e.g., high usage of rare codons, etc. are optionally screened/characterized for such gene's involvement in virulence or pathogenesis. The identified areas (i.e., the identified genes) are then optionally tested/screened for involvement in virulence or pathogenesis. Numerous methods of analysis to determine whether identified putative virulence genes are actually involved in virulence are known to those skilled in the art. Additionally, common sources of information for such determination include, e.g., Berger and Kimmel, Guide to Molecular Cloning Techniques, Methods in Enzymology volume 152 Academic Press, Inc., San Diego, Calif. (“Berger”); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). Additionally, U.S. patent applications U.S. Ser. No. 09/792,437 (filed Feb. 23, 2001) and U.S. Ser. No. 09/792,878 (filed Feb. 23, 2001), as well as PCT publications PCT/US01/05920 and PCT/US01/05955 detail comparable screens which are optionally adaptable to the current invention (e.g., such screens can optionally be used to test for or verify a virulence gene and/or gene product identified through use of the methods herein). Such sources (as well the references cited therein) are incorporated herein for all purposes.

Possible means of screening the phenotypic virulence contribution of any putative virulence genes identified through the methods of the invention include, e.g., sense/antisense screening, knockout screenings, homologous recombination, introduction of the putative virulence gene into a non-virulent strain (and/or introduction of the putative virulence gene under a controllable promoter into a virulent strain), etc. Again, such techniques are well known to those skilled in the art, and further information on such techniques is available in, e.g., Ausubel, Sambrook, Berger, etc., supra. Thus, some embodiments of the methods of the present invention provide methods for screening a gene product for its involvement in virulence. The gene product can be, e.g., a protein (for example, an enzyme), a ribonucleic acid sequence (such as a ribozyme), or a deoxyribonucleic acid sequence, etc. The gene that encodes the gene product used in the methods can be a gene present in the cellular genome, or it can be a gene present in a structure external to the cellular genome, such as a virus, a plasmid, a PAI, an expression vector and the like.

In preparing the screen, cells (if utilized) can be treated such that the expression level of the screened gene product (e.g., the putative virulence gene product) is altered. Manipulation of the expression of the gene product can be performed at the level of the gene or at the level of the gene product. For example, the expression of gene product can be controlled at the gene level through, e.g., stimulation or inhibition of various transcription activities, alteration of promoters, generation of temperature sensitive mutations and the like. Production of the gene product can be influenced by the levels of translation factors available, by the presence of transcript-specific ribozymes, or using anti-sense technology. The putative virulence activity of the gene product can be directly affected by addition of inhibitors or enhancers. Thus, the method used to manipulate the gene product can vary from assay to assay, depending upon the compound to be assayed and the gene product involved.

For example, in some embodiments, ribozymes (e.g., short RNA molecules having an antisense sequence and endoribonuclease activity which cleave other RNA molecules based on sequence specificity) are utilized to destroy functional expression by putative virulence genes (by cleaving the relevant expressed RNA). One class of ribozymes is derived from a number of small circular RNAs which are capable of self-cleavage and replication. General methods for the construction of ribozymes, including, e.g., hairpin ribozymes, hammerhead ribozymes, RNAse P ribozymes (i.e., ones derived from naturally occurring RNAse P ribozyme from prokaryotes or eukaryotes) are well to those skilled in the art. See also, e.g., Castanotto et al. (1994) Advances in Pharmacology 25:289 which provides an overview of ribozymes in general.

Antisense RNA molecules have long been known to inhibit expression of selected genes. Thus, they too are optionally used to verify involvement of identified genes in virulence. A number of references describe antisense and sense suppression, including, e.g., Antisense Strategies, Annals of the New York Academy of Sciences, Volume 600, Baserga and Denhardt (eds.) (NYAS 1992); Milligan et al., (1993) J Med Chem 36(14):1923-1937; Antisense Research and Applications (1993, CRC Press), Antisense Therapeutics, Sudhir Agrawal (ed.) (Humana Press, Totowa, N.J., 1996); and U.S. Pat. No. 4,801,340. Furthermore, “sense suppression” of genes has also been observed. For examples of the use of sense suppression to modulate expression of endogenous genes has also been observed, e.g., those genes identified herein as putatively involved in virulence, see, Napoli, et al., The Plant Cell (1990) 2:279 and U.S. Pat. No. 5,034,323.

Other means of verification that putative virulence genes are involved in virulence include use of DNA or RNA molecules that act as decoy nucleic acids, i.e., nucleic acids having a sequence recognized by a regulatory nucleic acid binding protein (e.g., a transcription factor, cell trafficking factor, etc.). Upon expression, the transcription factor binds to the decoy nucleic acid, rather than to its natural target in the genome (i.e., the putative virulence gene product).

In other embodiments, the sequences of interest (i.e., the putative virulence gene, etc.) can be selected based on well established methods such as traditional mutagenesis analysis, and reverse genetics methods such as gene knockouts. In summary, many techniques are available to verify whether identified sequences/genes are indeed involved in virulence/pathogenesis.

In some embodiments, various tRNA species are identified, e.g., in embodiments of methods of regulating gene expression in a bacterial organism, etc. For example, identification of at least one tRNA species responsible for encoding at least one member of one or more over/under represented codons, etc is included herein. Such tRNA species are identified (and modulators of such are also identified) through any number of well known screens and assays. For example, U.S. patent applications U.S. Ser. No. 09/792,437 (filed Feb. 23, 2001) and U.S. Ser. No. 09/792,878 (filed Feb. 23, 2001), as well as PCT publications PCT/US01/05920 and PCT/US01/05955 detail various screens which are optionally adaptable to such uses. Thus, such references (as well the references cited therein) are incorporated herein for all purposes. Additionally, further information is found in “Comparative Genomic Analysis of An Obligate Intracellular Taxon: Chlamydia trachomatis and Chlamydia pneumoniae” by Wayne P. Mitchell, Dissertation U.C. Berkeley (1999), which is incorporated herein by reference for all purposes, as are the references contained therein.

Identification/screening for Compounds that Effect Virulence Gene Products Identified Through the Methods of the Invention

In other embodiments of the invention, virulence genes (e.g., those identified through the methods herein based upon, e.g., concentration of rare codon usage and/or those screened for actual impact on virulence) are optionally screened to identify and/or isolate one or more modulator/inhibitor of such virulence genes. Again, screens for such modulators are well known to those in the art (e.g., high throughput screening of such things as commercial/public libraries of peptides, nucleic acids, chemical entities, etc. though use of e.g., microtiter plates, robots, microfluidics, etc.). For example, U.S. patent applications U.S. Ser. No. 09/792,437 (filed Feb. 23, 2001) and U.S. Ser. No. 09/792,878 (filed Feb. 23, 2001), as well as PCR publications PCT/US01/05920 and PCT/US01/05955 detail comparable screens which are optionally adaptable to the current invention (e.g., such screens can optionally be used to test for modulators, inhibitors, etc. of any virulence gene product identified through use of the methods herein).

For example, in some embodiments, the present invention comprises methods entailing screening of large libraries (e.g., chemical libraries). Such libraries can optionally include a wide variety of different compounds, including chemical compounds, mixtures of chemical compounds, polysaccharides, small organic or inorganic molecules, biological macromolecules (e.g., such as peptides, proteins, nucleic acids, etc.), extracts made from biological materials such as bacteria, plants, fungi, or animal cells or tissues, naturally occurring or synthetic compositions, etc. Typically such libraries can have in excess of 1,000, 10,000, or even 100,000 or more constituents. In other typical embodiments, the screening of libraries (e.g., to identify compounds/molecules that affect virulence genes and/or virulence gene products that are identified in other methods herein) is performed in a high throughput manner. See, below. Additionally, such screenings are optionally carried out with ancillary devices, such as, e.g., robots (e.g., used in plate handling, sample mixing, etc.), microtiter plates, or microfluidic devices (see, below).

The screening of compounds which putatively attenuate virulence (e.g., screening of large libraries or combinatorial libraries) is optionally carried out in vivo (e.g., the putative attenuators are inserted, uptaken, or transferred, etc. into a cell), or in vitro, e.g., the putative attenuators are screened against an, e.g., cell lysate or n a cell free system (depending upon, e.g., which specific virulence genes, etc. are being attenuated or possibly attenuated) by the putative attenuators.

High Throughput Methodology

As is apparent from the foregoing, the relevant assays of the invention will depend on the specific molecules/genes being screened and/or identified. Many assay formats are suitable for many applications. Advantageously, the assays optionally can be practiced in a high-throughput format. Optionally, one or more of any of the screenings, characterizations, identifications, or the like utilized herein can be employed in a rapid analysis system. For example, techniques for the growth of bacteria, etc., in multi-well plates and transformation of cells within multi-well plates are well known to those skilled in the art. Such methods are optionally employed in the techniques herein.

In high throughput assays, it is possible to screen up to several thousand different variants (e.g., different putative modulators of virulence gene products) in a single day. For example, each well of a microtiter plate can be used to run a separate assay, or, if concentration or incubation time effects are to be observed, every 5-10 wells can test a single variant. Thus, a single standard microtiter plate can assay about 100 (e.g., 96) different reactions. If 1536 well plates are used, then a single plate can easily accommodate from about 100 to about 1500 different reactions; it is possible to assay several different plates per day. Assay screens for up to about 6,000-20,000 different assays, (i.e., involving different nucleic acids, encoded proteins, concentrations, etc.) can also be used. Microfluidic approaches to reagent manipulation also have been developed and are optionally used in the methods herein, e.g., by Caliper Technologies (Mountain View, Calif.).

Molecules involved in modulation of virulence (e.g., molecules involved in modulation of tRNAs associated with rare codons present in virulence genes) can be prepared and screened in parallel fashion for, e.g., mass spectroscopy, LC/MS, LC-NMR, or any other appropriate analytical instrumentation in a parallel fashion using multi-well plates. Multi-well plates having 96, 384, 768, or 1536 or more wells are available from a number of commercial suppliers (e.g., VWR Scientific Products, West Chester, Pa.), as are the instrumentation for, e.g., autosampling from such plates, transfer to and from such plates, etc. Thus, by using a multi-well format, the methods of the present invention can be performed in a parallel high throughput manner.

Therapeutic Usage

In yet other embodiments herein, any modulator/inhibitor of an identified virulence gene (and/or gene product depending upon context herein) is optionally used as a prophylactic and/or therapeutic agent to treat a subject against a virulent/pathogenic organism comprising the identified virulence gene/gene product.

In some embodiments, compounds/molecules, etc. identified through the screening methods herein are optionally used to therapeutically and/or prophylactically treat subjects in order to, e.g., attenuate the virulence of pathogenic organisms (e.g., typically bacteria). Such compounds/molecules, etc. which attenuate the virulence of the pathogenic organisms are optionally injected parenterally, (e.g., intravenously, intraperitoneally, intramuscularly, or subcutaneously, etc.) in a subject. In other embodiments, the compositions of the invention are delivered via non-injection means, such as through oral means (e.g., pills, liquids, etc.), nebulized, etc. Various delivery systems for therapeutic treatments are well known to those skilled in the art.

Typically, the dosage ranges for such administration are large enough to elicit the desired effect in the subject (e.g., attenuation of virulence in the pathogenic organism in the host). The dosages given are optionally optimized for the individual subject based upon, e.g., the subject's age, gender, species, and weight, as well the presence of the pathogen. Doses are optionally given in a series. In other words, multiple doses are optionally given over a course of treatment. The dosage course is optionally modified during the treatment based upon the subject's (i.e., host's) response and/or the response of the pathogen (e.g., the response of the pathogenic bacteria, etc.). For example, if a subject does not response satisfactorily within a specific time period and/or if the pathogenic organism does not respond with attenuation of virulence, the dosage and/or timing of dosages is optionally increased or altered.

The present invention also includes methods of therapeutically or prophylactically treating the presence of a pathogenic organism, by administering in vivo or ex vivo one or more nucleic acids or polypeptides as described herein, e.g., biological compounds that act to attenuate the virulence of a pathogenic organism (or compositions comprising a pharmaceutically acceptable excipient and one or more such nucleic acids or polypeptides and/or fusion proteins) to a subject, including, e.g., a mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or commercially important invertebrate.

In each of the in vivo and ex vivo treatment methods, a composition comprising an excipient and the compound that attenuates the virulence of the pathogen or a nucleic acid encoding such compound, etc. can be administered or delivered. In one aspect, a composition comprising a pharmaceutically acceptable excipient and such molecules or nucleic acid is administered or delivered to the subject in an amount effective to treat the disease or disorder (e.g., by attenuating the virulence of the pathogen).

Digital Systems

The present invention provides digital systems, e.g., computers, computer readable media and integrated systems comprising the equations/calculations/etc.herein. Various methods known in the art can be used to perform the calculations herein or to detect, e.g., open reading frame, codons (e.g., proper reading frames, etc.), or to perform other desirable functions such as to control output files, provide the basis for making presentations of information including sequences and the like. Computer systems of the invention can include such programs, e.g., in conjunction with one or more data file or data base comprising sequences as noted herein.

Thus, standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™, Paradox™, GeneWorks™, or MacVector™) can be adapted to the present invention by inputting a codon strings corresponding to one or more, e.g., pathogenic organism. For example, a system of the invention can include the foregoing software having the appropriate codon string information, e.g., used in conjunction with a user interface (e.g., a GUI in a standard operating system such as a Windows, Macintosh or LINUX system) to manipulate strings of characters corresponding to the sequences herein.

Systems in the present invention typically include a digital computer with data sets entered into the software system comprising any of the calculations, etc. herein. The computer can be, e.g., a PC (Intel×86 or Pentium chip-compatible DOS™, OS2™ WINDOWS™ WINDOWSNT™, WINDOWS95™, WINDOWS2000™, WINDOWS98™, LINUX based machine, a MACINTOSH™, Power PC, or a UNIX based (e.g., SUN™ work station) or other commercially common computer that is known to one of skill. Software for performing the analyses, herein or otherwise manipulating, e.g., codon sequences is available, or can easily be constructed by one of skill using a standard programming language such as Visualbasic, PERL, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation, e.g., of appropriate calculations to determine CDI, etc.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of determining a difference in codon usage between a selected nucleic acid sequence and a reference genome, the method comprising:
(a) selecting a codon i from a set of n codons;
(b) determining the number of occurrences of codon i in the selected nucleic acid sequence and in the reference genome;
(c) calculating a first occurrence frequency fᵢ, wherein
$${f}_{i} = \frac{( \# {codon}_{i} ) ( 1000 codons )}{( \# codons\_in \_all \_reference \_genome \_orfs )}$$
(d) calculating a second occurrence frequency cᵢ, wherein
$${c}_{i} = \frac{( \# {codon}_{i} ) ( 1000 codons )}{( \# codons\_in \_selected \_sequence )}$$
(e) calculating an average difference CDI between the first occurrence frequency fᵢ and second occurrence frequency cᵢ, wherein
$$CDI = \frac{{\sum }_{i - 1}^{n} \vert {c}_{i} - {f}_{i} \vert}{n}$$
and wherein a value of CDI indicates the difference in usage of codon i in the selected nucleic acid sequence as compared to the reference genome.

2. The method of claim 1, wherein the set of n codons comprises 61 non-stop codons.

3. The method of claim 1, wherein the set of n codons comprises a set of rare codons in the reference genome.

4. The method of claim 3, wherein the set comprises the 10 rarest codons in the reference genome.

5. The method of claim 1, wherein the first occurrence frequency fᵢ is calculated only with reference to ORFs comprising about 250 or more amino acids.

6. A method of identifying a putative target for attenuation of pathogen virulence, the method comprising:
(a) determining a codon usage frequency of one or more codon of a pathogen;
(b) identifying at least one gene comprising one or more over-represented codon or one or more under-represented codon;
(c) identifying a set of tRNA molecules responsible for interacting with the one or more over-represented codon or under-represented codon in the at least one gene during translation;
(d) providing a population of nucleic acid sequences encoding a putative target for attenuation of pathogenic virulence and an in vitro or in vivo translation system;
(e) altering a translation process involving one or more member of the set of tRNA molecules and the in vitro or in vivo translation system, thereby altering expression of at least one member of the population in (d); and,
(f) testing for one or more effect of the altering, thereby identifying one or more putative target for attenuation of pathogen virulence.

7. The method of claim 6, wherein altering the translation process comprises preventing the one or more members of the set of tRNA molecules from interacting with an mRNA encoding the putative target.

8. The method of claim 6, wherein altering the translation process comprises interfering with a process for synthesizing one or more members of the set of tRNA molecules.

9. The method of claim 8, wherein interfering with synthesizing the tRNA molecule comprises altering a base modification in a tRNA sequence.

10. The method of claim 6, wherein altering the translation process comprises altering the translation efficiency or accuracy of one or more member of the set of tRNA molecules.

11. The method of claim 6, further comprising screening one or more compositions for one or more virulence modulatory effect on the target.

12. The method of claim 11, wherein the screening comprises 1,000 or more compositions.

13. The method of claim 12, wherein the screening comprises 5,000 or more compositions.

14. The method of claim 13, wherein the screening comprises 10,000 or more compositions.

15. A method of identifying virulence-related nucleic acid sequences in a pathogenic organism, the method comprising:
(a) analyzing a population of nucleic acid sequences derived from the pathogenic organism and identifying one or more over-represented codons or under represented codons as compared to a nonpathogenic organism;
(b) determining a distribution for at least one member of the one or more over-represented codons or under-represented codons;
(c) selecting a subset of nucleic acid sequences from the population of nucleic acid sequences based upon the distribution of the over-represented or under-represented codons; and,
(d) analyzing the subset of nucleic acid sequences for virulence activity, thereby identifying one or more virulence-related nucleic acid sequence in a pathogenic organism.

16. The method of claim 15, wherein the subset of nucleic acid sequences is selected based upon a number of over-represented codons in that nucleic acid sequence.

17. The method of claim 15, wherein the subset of nucleic acid sequences is selected based upon a number of under-represented codons in that nucleic acid sequence.

18. The method of claim 15, wherein the nonpathogenic organism and the pathogenic organism are different serovars of a common ancestral organism.

19. The method of claim 15, wherein the pathogenic organism and the nonpathogenic organism are two strains of the same species.

20. The method of claim 15, wherein the nonpathogenic organism is E. coli K12 and the pathogenic organism comprises one or more of E. coli O157:H7, E. coli 171, or Shigella flexneri.

21. The method of claim 15, wherein the virulence-related nucleic acid sequence comprises one or more tRNA molecule responsible for encoding the at least one member of the one or more over-represented codons or under-represented codons.

22. The method of claim 21, further comprising:
(e) identifying one or more structural characteristics of the one or more tRNA molecule; and,
(f) modulating the activity of the one or more tRNA molecules.

23. The method of claim 15, wherein the virulence-related nucleic acid sequence comprises one or more tRNA synthase molecule.

24. The method of claim 15, further comprising screening one or more compositions for one or more virulence-related nucleic acid sequences.

25. The method of claim 24, wherein the screening comprises 1,000 or more compositions.

26. The method of claim 25, wherein the screening comprises 5,000 or more compositions.

27. The method of claim 26, wherein the screening comprises 10,000 or more compositions.

28. The method of claim 23, further comprising: identifying one or more structural characteristics of the one or more tRNA synthase molecule; and, modulating the activity of the one or more tRNA synthase molecule.

29. A method of regulating gene expression in a bacterial organism, the method comprising:
(a) identifying one or more over-represented codons or under-represented codons within a set of nucleic acid sequences from a bacterial organism;
(b) identifying at least one tRNA species responsible for encoding at least one of the one or more over-represented codons or under-represented codons; and,
(c) modulating an expression or activity of the at least one tRNA species in the bacterial organism; thus, altering a translation of a nucleic acid sequence comprising the one or more over-represented or under-represented codons, thereby regulating the expression of one or more gene in the bacterial organism.

30. The method of claim 29, wherein identifying the one or more over-represented codons or under-represented codons comprises determining a distribution for at least one member of the one or more over-represented codons or under-represented codons.

31. The method of claim 29, wherein the set of nucleic acid sequences from the bacterial organism comprises a library of mRNA sequences.

32. The method of claim 29, wherein the set of nucleic acid sequences from the bacterial organism comprises sequences from one or more pathogenicity islands.

33. The method of claim 29, wherein identifying the at least one tRNA species comprises:
(a) measuring the codon usage of each gene in the bacterial organism;
(b) cataloging the at least one tRNA gene in the bacterial organism; and,
(c) detecting one or more modification in the tRNA which will modulate expression of one or more gene in the bacterial genome wherein the one or more gene is over-represented in a particular codon.

34. The method of claim 33, wherein the measuring comprises use of a counting algorithm.

35. The method of claim 34, wherein the algorithm comprises PERL language code.

36. The method of claim 33, wherein the cataloging comprises use of tRNAscan-SE software.

37. The method of claim 33, wherein detecting one or more modification in the tRNA comprises use of one or more of: cognate codon-anticodon interactions or codon-anticodon wobble rules.

38. The method of claim 29, wherein modulating the expression or activity of the at least one tRNA species comprises reducing an extent of diversity of the tRNA species.

39. The method of claim 29, wherein modulating the expression or activity of the at least one tRNA species comprises altering a chemical character or chemical characteristic of the tRNA species.

40. The method of claim 29, wherein modulating the expression or activity of the at least one tRNA species comprises inhibiting a tRNA modification synthase activity specific for that at least one tRNA species.

41. The method of claim 29, wherein modulating the expression or activity of the at least one tRNA species comprises inhibiting an interaction between the tRNA species and an additional RNA molecule.

42. The method of claim 41, wherein the additional RNA molecule comprises an mRNA molecule.

43. The method of claim 41, wherein the additional RNA molecule comprises an rRNA molecule.

44. The method of claim 29, altering the translation of the nucleic acid sequence comprises inhibiting the translation of an mRNA molecule

45. The method of claim 29, wherein altering the translation of the nucleic acid sequence comprises enhancing the translation of an mRNA molecule.

46. The method of claim 29, further comprising screening one or more compositions for one or more compound that modulates expression or activity of the at least one tRNA species.

47. The method of claim 46, wherein screening comprises 1,000 or more compositions.

48. The method of claim 47, wherein screening comprises 5,000 or more compositions.

49. The method of claim 48, wherein screening comprises 10,000 or more compositions.

50. A method of attenuating the virulence of a pathogenic organism, the method comprising:
(a) identifying one or more tRNA species encoding one or more over-represented codons within a set of virulence-related nucleic acid sequences from a bacterial organism, wherein the over-represented codon is over-represented in relation to a usage of the codon in the rest of the genome;
(b) inhibiting an in vivo expression or activity of the tRNA species within the bacterial organism, thereby decreasing the virulence of the pathogenic organism.

51. The method of claim 50, wherein identifying the one or more tRNA species comprises:
(a) measuring the codon usage of each gene in the bacterial organism;
(b) cataloging the at least one tRNA gene in the bacterial organism; and,
(c) detecting one or more modification in the tRNA which will modulate expression of one or more gene in the bacterial genome wherein the one or more gene is over-represented in a particular codon.

52. The method of claim 50, wherein inhibiting the in vivo expression or activity of the tRNA species comprises reducing an extent of diversity of the tRNA species.

53. The method of claim 50, wherein inhibiting the in vivo expression or activity of the tRNA species comprises inhibiting a tRNA synthase activity specific for the one or more tRNA species.

54. The method claim 50, wherein inhibiting the in vivo expression or activity of the tRNA species comprises inhibiting an interaction between the tRNA species and an additional RNA molecule.

55. A method for selectively affecting one or more pathogenic organism in a population, the method comprising:
(a) providing a first population comprising nucleic acid sequences from a pathogenic organism;
(b) providing a second population comprising nucleic acid sequences from a nonpathogenic organism, which nonpathogenic organism comprises a same species as the pathogenic organism;
(c) determining a distribution of codon usage in the pathogenic organism as compared to a distribution of a codon usage in the nonpathogenic organism; and,
(d) selecting one or more codons that are over-represented or under-represented in the nucleic acid sequences of the pathogenic organism based upon the distribution of codon usage in the pathogenic organism and the nonpathogenic organism,
(e) identifying at least one tRNA species responsible for encoding at least one selected codon, which selected codon comprises a codon that is over-represented or under-represented in the pathogenic organism relative to the nonpathogenic organism; and,
(f) altering the expression or activity of the identified tRNA species, thereby selectively affecting the pathogenic organisms in the population.

56. The method of claim 55, wherein altering comprises identifying one or more structural characteristics of the at least one tRNA species; and, providing an antibody specific to the at least one tRNA, which antibody binds to the tRNA, thus preventing an action by the tRNA.

57. The method of claim 55, wherein altering comprises identifying one or more enzymes for synthesizing the one or more tRNA species; and, inhibiting the one or more enzymes.
